(19) United States
(12) Patent Application Publication
(10) Pub. No.: US 2020/0239564 A1
(43) Pub. Date: Jul. 30, 2020

(54) USE OF CANAKINUMAB

(71) Applicant: NOVARTIS AG, Basel (CH)

(72) Inventors: Paul RIDKER, Chestnut Hill, MA (US); Tom THUREN, Succasunna, NJ (US); Georgina BERMANN, Basel (CH); Peter LIBBY, Boston, MA (US)

(21) Appl. No.: 16/641,889

(22) PCT Filed: Aug. 24, 2018
(86) PCT No.: PCT/IB2018/056465

§ 371 Date: Feb. 25, 2020

Related U.S. Application Data
(60) Provisional application No. 62/549,971, filed on Aug. 25, 2017.
(60) Provisional application No. 62/584,380, filed on Nov. 10, 2017.

(51) Int. Cl. C07K 16/24; A61P 9/10

(52) U.S. Cl. CPC C07K 16/245; A61K 2039/505; A61P 9/10

(57) ABSTRACT

The present invention relates to canakinumab for use in reducing the risk of or preventing recurrent cardiovascular (CV) events in a patient with elevated hsCRP that has suffered myocardial infarction (MI).

### TECHNICAL FIELD

The present disclosure relates to novel uses and methods for reducing the risk of or preventing recurrent cardiovascular (CV) events in a patient that has suffered myocardial infarction (MI), comprising administering canakinumab.

### BACKGROUND OF THE DISCLOSURE

Atherothrombosis is characterized by atherosclerotic lesion disruption with superimposed thrombus formation and is the major cause of acute coronary syndromes (ACS) and cardiovascular death. Atherothrombosis is the leading cause of mortality in the industrialized world. Arterial inflammation and endothelial dysfunction play key roles at all stages of the atherothrombotic process. Inflammatory mediators are intimately implicated with the cascade of events leading to atherosclerotic plaque initiation, progression and rupture. Vascular endothelial cells express a variety of adhesion molecules that recruit monocytes when chronically exposed to noxious stimuli or pathological conditions. Adverse conditions such as hyperlipidemia are associated with enrichment of a pro-inflammatory subset of monocytes. These monocytes apparently enter the intima under the influence of chemotactic stimuli and engulf modified low density lipoprotein (LDL) and cholesterol crystals (Duewell P et al, Nature. 2010; 464(7293):1357-61). The material internalized by phagocytes induces phagolysosomal damage and subsequent leakage of contents into cytosol to activate inflammasomes and caspase 1, and consequently the generation of interleukin-1β (IL-1β) from pro-interleukin-1β.

Interleukins are key mediators in the chronic vascular inflammatory response in cardiovascular (CV) disease and have been demonstrated in animal models and in humans to be potent modulators of pro-inflammatory processes. The fact that these cytokines and their receptors are highly expressed and are functional in almost all cell types implicated in the pathogenesis of atherosclerosis including smooth muscle cells, certain subset of macrophages and T cells as well as endothelium supports the role of interleukins in vascular disease. This concept is further supported by the notion that despite the success of statin therapy in reducing hyperlipidemia and thereby lowering the risk of myocardial infarction, stroke and cardiovascular death, many post-myocardial infarction patients receiving statin therapy continue to suffer from life threatening vascular events. This high risk for recurrent cardiovascular events despite the use of aggressive secondary prevention strategies is at least partly due to residual inflammation (Ridker P M. Eur Heart J. 2016; 37(22):1720-2). Thus, novel therapies that decrease inflammation, improve vascular function, decrease atherosclerotic burden, and ultimately translate to a decrease in cardiovascular events fill a significant unmet medical need.

### SUMMARY OF THE DISCLOSURE

Inflammation contributes to all phases of the atherothrombotic process and patients with elevated inflammatory biomarkers such as hsCRP and IL-6 have increased vascular risk despite use of aggressive secondary prevention strategies. The present disclosure relates, in part, to the finding that direct inhibition of inflammation by administration of canakinumab reduces the risk of or prevents recurrence of cardiovascular events in post-myocardial infarction patients responding to canakinumab.

Accordingly, the present invention is directed to a method for reducing the risk of or preventing recurrent cardiovascular (CV) events in a patient that has suffered myocardial infarction (MI), comprising administering about 150 mg to about 300 mg of canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab.

The present invention is also directed to a method for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately 3 months after first administration of canakinumab.

Accordingly, the present invention is also directed to canakinumab for use in reducing the risk of or preventing recurrent cardiovascular (CV) events in a patient that has suffered myocardial infarction (MI),

wherein said patient has a high sensitivity C-reactive protein (hsCRP) level of ≥2 mg/L assessed at least 28 days after MI and before first administration of canakinumab, and

wherein about 150 mg to about 300 mg of canakinumab is administered to the patient at the earliest 30 days after MI, and

wherein said patient will continue to receive about 150 mg to about 300 mg canakinumab approximately every 3 months, and

wherein said patient has a reduced hsCRP level of <2 mg/L assessed approximately 3 months after first administration of canakinumab.

Accordingly, the present invention is also directed to the use of canakinumab in reducing the risk of or preventing recurrent cardiovascular (CV) events in a patient that has suffered myocardial infarction (MI), wherein
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed approximately 3 months after first administration of canakinumab.

The present invention is further directed to the canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

The present invention is also directed to the use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed approximately 3 months after first administration of canakinumab.

Further features and advantages of the disclosure will become apparent from the following detailed description of the invention.

### BRIEF DESCRIPTION OF THE DRAWINGS

FIG. 1. Effects of canakinumab as compared to placebo on plasma levels of high-sensitivity C-reactive protein (hsCRP), low-density lipoprotein (LDL) cholesterol, high-density lipoprotein (HDL) cholesterol, and triglycerides during trial follow-up. Data are shown as median percent change from baseline. Specific data points at 3 months, 12 months, 24 months, 36 months and 48 months as well as data points for interleukin-6 (IL-6) at 3 months and 12 months are presented in Tables 2 to 6.

FIG. 2. Panels A-C: Cumulative incidence of the trial primary end point of nonfatal myocardial infarction, nonfatal stroke, or cardiovascular death in the placebo and canakinumab 50 mg group (Panel A), 150 mg group (Panel B), and 300 mg group (Panel C). Panels D-F: Cumulative incidence of the trial secondary end point (primary cardiovascular end point plus hospitalization for unstable angina requiring urgent revascularization) in the placebo and canakinumab 50 mg group (Panel D), 150 mg group (Panel E), and 300 mg group (Panel F).

FIG. 3. CANTOS Trial Diagram.

FIG. 4. Effects of placebo and canakinumab on hsCRP, IL-6, and lipids 3 months after first dose of canakinumab. LDLC=low-density lipoprotein cholesterol, HDLC=high-density lipoprotein cholesterol, TG=triglycerides.

FIG. 5. Cumulative incidence of the primary cardiovascular endpoint in the combined 150 mg and 300 mg groups (150 mg/300 mg). SC=subcutaneous, q 3 months=every three months.

FIG. 6. Cumulative incidence of the secondary cardiovascular endpoint in the combined 150 mg and 300 mg groups (150 mg/300 mg). SC=subcutaneous, q 3 months=every three months.

FIG. 7. Clinical efficacy of canakinumab as compared to placebo for the trial primary endpoint (nonfatal myocardial infarction, nonfatal stroke, or cardiovascular death, left) and the trial secondary endpoint (nonfatal myocardial infarction, nonfatal stroke, hospitalization for unstable angina requiring unplanned revascularization, or cardiovascular death, right) according to prespecified subgroups based upon baseline clinical characteristics. LDLC=low-density lipoprotein cholesterol, HDLC=high-density lipoprotein cholesterol, hsCRP=high-sensitivity C-reactive protein, TG=triglycerides.

FIG. 8. Cumulative incidence of cardiovascular events in CANTOS in the placebo group and in the combined canakinumab groups according to whether 3 month on-treatment hsCRP levels were above or below the commonly used clinical cutpoint of 2 mg/L. Data are shown for the trial primary endpoint (nonfatal myocardial infarction, nonfatal stroke, or cardiovascular death).

FIG. 9. Cumulative incidence of cardiovascular events in CANTOS in the placebo group and in the combined canakinumab groups according to whether 3 month on-treatment hsCRP levels were above or below the commonly used clinical cutpoint of 2 mg/L. Data are shown for the key pre-specified secondary endpoint (nonfatal myocardial infarction, nonfatal stroke, hospitalization for unstable angina requiring unplanned revascularization, or cardiovascular death).

FIG. 10. Cumulative incidence of the CANTOS primary endpoint (nonfatal myocardial infarction, nonfatal stroke, or cardiovascular death) in the placebo group and in the combined canakinumab groups according to tertiles of on-treatment hsCRP achieved at 3 months after the initial dose of canakinumab.

FIG. 11. Cumulative incidence of the CANTOS pre-specified key secondary cardiovascular endpoint (nonfatal myocardial infarction, nonfatal stroke, cardiovascular death or unstable angina requiring urgent revascularization) in the placebo group and in the combined canakinumab groups according to tertiles of on-treatment hsCRP achieved at 3 months after the initial dose of canakinumab.

FIG. 12. Cumulative incidence of the CANTOS primary endpoint (nonfatal myocardial infarction, nonfatal stroke, or cardiovascular death) as observed for patients treated with 150 mg canakinumab and the average placebo curve derived using the covariates to predict the counterfactual placebo response of canakinumab patients in the hsCRP<1.5 mg/L and ≥1.5 mg/L subgroups achieved at 3 months after the initial dose of canakinumab.

FIG. 13. Cumulative incidence of the CANTOS primary endpoint (nonfatal myocardial infarction, nonfatal stroke, or cardiovascular death) as observed for patients treated with 150 mg canakinumab and the average placebo curve derived using the covariates to predict the counterfactual placebo response of canakinumab patients in the hsCRP<1.8 mg/L and ≥1.8 mg/L subgroups achieved at 3 months after the initial dose of canakinumab.

FIG. 14. Cumulative incidence of the CANTOS primary endpoint (nonfatal myocardial infarction, nonfatal stroke, or cardiovascular death) as observed for patients treated with 150 mg canakinumab and the average placebo curve derived using the covariates to predict the counterfactual placebo response of canakinumab patients in the hsCRP<2 mg/L and ≥2 mg/L subgroups achieved at 3 months after the initial dose of canakinumab.

### DETAILED DESCRIPTION OF THE DISCLOSURE

The present invention provides, inter alia, methods for reducing the risk of or preventing recurrent cardiovascular (CV) events in a patient that has suffered myocardial infarction (MI), comprising administering about 150 mg to about 300 mg of canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab.

The present invention also provides methods for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately three months after first administration of canakinumab.

The present invention provides canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

The present invention also provides the use of canakinumab in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed approximately 3 months after first administration of canakinumab.

The present invention provides the use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

The present invention also provides the use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and wherein
ii) about 150 mg to about 300 mg of canakinumab is administered to the patient at the earliest 30 days after MI, and wherein
iii) said patient will continue to receive about 150 mg to about 300 mg canakinumab approximately every 3 months, provided said patient has a reduced hsCRP level of <2 mg/L assessed approximately 3 months after first administration of canakinumab.

The present invention arose from the analysis of the data generated from the CANTOS trial (Ridker P M et al, Am Heart J. 2011; 162(4):597-605 and as disclosed in WO2013/049278, which is hereby incorporated by reference in its entirety), a randomized, double-blind, placebo-controlled, event-driven trial, designed to evaluate whether the administration of quarterly subcutaneous canakinumab can prevent recurrent cardiovascular events among stable post-myocardial infarction patients with elevated hsCRP. The enrolled 10,061 patients with myocardial infarction and inflammatory atherosclerosis had high sensitivity C-reactive protein (hsCRP) of ≥2 mg/L. Three escalating canakinumab doses (50 mg, 150 mg, and 300 mg given subcutaneously every 3 months) were compared to placebo.

Canakinumab (international nonproprietary name (INN) number 8836) is disclosed in WO02/16436, which is hereby incorporated by reference in its entirety. Canakinumab is a fully human monoclonal anti-human IL-1β antibody of the IgG1/k isotype, being developed for the treatment of IL-1β driven inflammatory diseases. It is designed to bind to human IL-1β, and thereby blocking the interaction of the cytokine with its receptors. The antagonism of the IL-1β mediated inflammation using canakinumab in lowering high sensitivity C-reactive protein (hsCRP) and other inflammatory marker levels has shown an acute phase response in patients with Cryopyrin-Associated Periodic Syndrome (CAPS) and rheumatoid arthritis. This evidence has been replicated in patients with type 2 diabetes mellitus (T2DM) using canakinumab and with other IL-1β antibody therapies in development, although in T2DM reduction in hsCRP levels did not translate to increased efficaciousness over standard of care treatment. IL-1β inhibition over a longer period of time, thereby inhibiting a major inflammatory pathway, will have unforeseen effects, which may be advantageous or not, therefore necessitating a large, randomized, placebo-controlled clinical trial monitoring multiple parameters.

The inventors have now found that treatment with canakinumab significantly reduces the risk of experiencing recurrent cardiovascular events in stable post-myocardial patients with elevated hsCRP by lowering residual inflammatory risk through administration of canakinumab without effecting the levels of HDL cholesterol, LDL cholesterol and triglycerides.

In one embodiment, the present invention provides a method for reducing the risk of or preventing recurrent cardiovascular (CV) events in a patient that has suffered myocardial infarction (MI), comprising administering about 150 mg to about 300 mg of canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab. In one embodiment, the present disclosure provides a method for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately 3 months after first administration of canakinumab. In one embodiment, any method of the invention comprises administering about 150, 175, 200, 225, 250, 275, 300 mg or any combination thereof of canakinumab.

One embodiment of any method of the invention comprises administering 150 mg canakinumab or 300 mg canakinumab. A particularly preferred embodiment of any method of the invention comprises administering 150 mg canakinumab. In a preferred embodiment of any method described herein, canakinumab is administered at the earliest 30 days after MI.

In one embodiment of any method described herein, said patient has high sensitivity C-reactive protein (hsCRP) levels of ≥3 mg/L assessed at least 28 days after MI and before first administration of canakinumab. In one embodiment of any method described herein, said patient has high sensitivity C-reactive protein (hsCRP) levels of ≥4 mg/L assessed at least 28 days after MI and before first administration of canakinumab. In one embodiment of any method described herein, said patient has high sensitivity C-reactive protein (hsCRP) levels of ≥5 mg/L assessed at least 28 days after MI and before first administration of canakinumab. In one embodiment of any method described herein, said patient has high sensitivity C-reactive protein (hsCRP) levels of ≥6 mg/L assessed at least 28 days after MI and before first administration of canakinumab. In one embodiment of any method described herein, said patient has high sensitivity C-reactive protein (hsCRP) levels of ≥7 mg/L assessed at least 28 days after MI and before first administration of canakinumab. In one embodiment of any method described herein, said patient has high sensitivity C-reactive protein (hsCRP) levels of ≥8 mg/L assessed at least 28 days after MI and before first administration of canakinumab. In one embodiment of any method described herein, said patient has high sensitivity C-reactive protein (hsCRP) levels of ≥9 mg/L assessed at least 28 days after MI and before first administration of canakinumab. In one embodiment of any method described herein, said patient has high sensitivity C-reactive protein (hsCRP) levels of ≥10 mg/L assessed at least 28 days after MI and before first administration of canakinumab.

In one embodiment of any method of the invention the reduced level of hsCRP assessed approximately 3 months after first administration of canakinumab is <1.9, <1.8, <1.7, <1.6, <1.5, <1.4, <1.3, <1.2, <1.1, <1.0, <0.9, <0.8, <0.7, <0.6, or <0.5 mg/L. In one embodiment, the reduced level of hsCRP assessed approximately 3 months after first administration of canakinumab is <1.8 mg/L. In another embodiment, the reduced level of hsCRP assessed approximately 3 months after first administration of canakinumab is <1.5 mg/L.

Accordingly, one embodiment of the present invention provides a method for reducing the risk of or preventing recurrent cardiovascular (CV) events in a patient that has suffered myocardial infarction (MI), comprising administering about 150 mg canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab. Accordingly, another embodiment of the present invention provides a method for reducing the risk of or preventing recurrent cardiovascular (CV) events in a patient that has suffered myocardial infarction (MI), comprising administering about 150 mg canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg canakinumab approximately every 3 months, provided said patient has a reduced hsCRP level of <2 mg/L assessed approximately 3 months after first administration of canakinumab.

Yet another embodiment of the present disclosure provides a method for reducing the risk of or preventing recurrent cardiovascular (CV) events in a patient that has suffered myocardial infarction (MI), comprising administering about 150 mg canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg canakinumab approximately every 3 months, provided said patient has a reduced hsCRP level of <1.8 mg/L assessed approximately 3 months after first administration of canakinumab.

Another embodiment provides a method for reducing the risk of or preventing recurrent cardiovascular (CV) events in a patient that has suffered myocardial infarction (MI), comprising administering about 150 mg canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg canakinumab approximately every 3 months, provided said patient has a reduced hsCRP level of <1.5 mg/L assessed approximately 3 months after first administration of canakinumab.

In one embodiment of any of the methods provided herein, the patient has reduced hsCRP level of <2 mg/L assessed approximately 3 months and approximately 6 months after first administration of canakinumab.

In another embodiment of any of the methods provided herein, the patient has reduced hsCRP level of <2 mg/L assessed approximately 3 months, approximately 6 months and approximately 9 months after first administration of canakinumab.

In another embodiment of any of the methods provided herein, the patient has reduced hsCRP level of <2 mg/L assessed approximately 3 months, approximately 6 months and approximately 9 months after first administration of canakinumab.

In another embodiment of any of the methods provided herein, the patient has reduced hsCRP level of <2 mg/L assessed approximately 3 months, approximately 6 months, approximately 9 months and approximately 12 months after first administration of canakinumab.

In another embodiment of any of the methods provided herein, the patient has reduced hsCRP level of <2 mg/L first assessed approximately 3 months after first administration of canakinumab and at approximately three-month intervals thereafter.

Accordingly, in one embodiment, provided is a method for reducing the risk of or preventing recurrent cardiovascular (CV) events in a patient that has suffered myocardial infarction (MI), wherein said patient has a high sensitivity C-reactive protein (hsCRP) level of ≥2 mg/L assessed at least 28 days after MI and before first administration of canakinumab, comprising a first administration of about 150 mg of canakinumab to said patient, and comprising further administration of about 150 mg of canakinumab approximately every 3 months, provided said patient has reduced hsCRP level of <2 mg/L assessed approximately 3 months and approximately 6 months after first administration of canakinumab.

In another embodiment, provided is a method for reducing the risk of or preventing recurrent cardiovascular (CV) events in a patient that has suffered myocardial infarction (MI), wherein said patient has a high sensitivity C-reactive protein (hsCRP) level of ≥2 mg/L assessed at least 28 days after MI and before first administration of canakinumab, comprising a first administration of about 150 mg of canakinumab to said patient, and comprising further administration of about 150 mg of canakinumab approximately every 3 months, provided said patient has reduced hsCRP level of <2 mg/L assessed approximately 3 months, approximately 6 months and approximately 9 months after first administration of canakinumab.

In another embodiment, provided is a method for reducing the risk of or preventing recurrent cardiovascular (CV) events in a patient that has suffered myocardial infarction (MI), wherein said patient has a high sensitivity C-reactive protein (hsCRP) level of ≥2 mg/L assessed at least 28 days after MI and before first administration of canakinumab, comprising a first administration of about 150 mg of canakinumab to said patient, and comprising further administration of about 150 mg of canakinumab approximately every 3 months, provided said patient has reduced hsCRP level of <2 mg/L assessed approximately 3 months, approximately 6 months, approximately 9 months and approximately 12 months after first administration of canakinumab.

In another embodiment, provided is a method for reducing the risk of or preventing recurrent cardiovascular (CV) events in a patient that has suffered myocardial infarction (MI), wherein said patient has a high sensitivity C-reactive protein (hsCRP) level of ≥2 mg/L assessed at least 28 days after MI and before first administration of canakinumab, comprising a first administration of about 150 mg of canakinumab to said patient, and comprising further administration of about 150 mg of canakinumab approximately every 3 months, provided said patient has reduced hsCRP level of <2 mg/L assessed approximately 3 months and approximately 9 months after first administration of canakinumab.

In one embodiment, also provided is a method for reducing the risk of or preventing recurrent cardiovascular (CV) events in a patient that has suffered myocardial infarction (MI), comprising administering about 150 mg of canakinumab approximately every 3 months, wherein said patient has a high sensitivity C-reactive protein (hsCRP) level of ≥2 mg/L assessed at least 28 days after MI and before first administration of canakinumab, and wherein said patient has reduced hsCRP levels of <2 mg/L first assessed approximately 3 months after first administration of canakinumab and at approximately three-month intervals thereafter.

In another embodiment, provided is a method for reducing the risk of or preventing recurrent cardiovascular (CV) events in a patient that has suffered myocardial infarction (MI), wherein said patient has a high sensitivity C-reactive protein (hsCRP) level of ≥2 mg/L assessed at least 28 days after MI and before first administration of canakinumab, comprising a first administration of about 150 mg of canakinumab to said patient, and comprising further administration of about 150 mg of canakinumab approximately every 3 months, provided said patient has reduced hsCRP levels of <2 mg/L first assessed approximately 3 months after first administration of canakinumab and at approximately three-month intervals thereafter.

As used herein, the term “recurrent CV events” is a repeated CV event taking place after the myocardial infarction qualifying the patient for treatment with canakinumab and is selected from non-fatal MI, non-fatal stroke, cardiovascular (CV) death and hospitalization for unstable angina requiring unplanned revascularization.

In one embodiment of any method of the invention, said recurrent CV event is selected from non-fatal MI, non-fatal stroke, cardiovascular (CV) death and hospitalization for unstable angina requiring unplanned revascularization. In another embodiment of any method of the invention, said recurrent CV event is selected from non-fatal MI, non-fatal stroke and cardiovascular (CV) death. In yet another embodiment of any method of the invention said recurrent CV event is non-fatal MI or cardiovascular (CV) death. In another embodiment of any method of the invention said recurrent CV event is non-fatal MI. In another embodiment of any method of the invention said recurrent CV event is hospitalization for unstable angina requiring unplanned revascularization.

In one embodiment the method of the invention optionally further comprises administering the patient an additional dose of 300 mg of canakinumab about two weeks (+/−3 days) from initial administration of canakinumab.

Accordingly, the present disclosure provides a method for reducing the risk of or preventing recurrent cardiovascular (CV) events in a patient that has suffered myocardial infarction (MI), comprising administering an initial dose of 300 mg of canakinumab, wherein said patient has a high sensitivity C-reactive protein (hsCRP) level of ≥2 mg/L assessed at least 28 days after MI and before the administration of canakinumab, and wherein canakinumab is administered at the earliest 30 days after MI, further comprising an additional dose of 300 mg of canakinumab two weeks from initial administration and wherein said patient will continue to receive subsequent doses of 150 mg or preferably 300 mg canakinumab about every 3 months, provided said patient has a reduced hsCRP level of <2 mg/L assessed at least three months after the initial administration of canakinumab.

In one aspect of the invention, the risk of experiencing recurrent CV events in a stable post-myocardial patient with hsCRP levels of ≥2 mg/L assessed at least 28 days after MI is reduced by 20% or 21% or 22% or 23% or 24% or 25% or 26% or 27% or 28% or 29% or 30% after administration comprising about 150 mg to about 300 mg of canakinumab.

In other embodiments of any method according to the invention, a biomarker other than hsCRP includes but is not limited to IL-6 (Ridker et al (2018) Eur Heart J, in press).

Other embodiments of the invention include the use of canakinumab according to any of the described uses or methods herein.

Other embodiments of the invention include:

Canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

Canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed approximately 3 months after first administration of canakinumab.

Use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

Use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed at least 3 months after first administration of canakinumab.

In the following pages, various aspects of the two uses stated in the four paragraphs above are described and all these aspects could be combined together. The skilled person realizes that the embodiments in the following pages are all combinable with each other and particular aspects combining features from various embodiments of these pages will be considered to be adequately disclosed to the skilled person.

In one embodiment, any use of the invention comprises administering about 150, 175, 200, 225, 250, 275, 300 mg or any combination thereof of canakinumab.

In one embodiment of any use described herein, said patient has high sensitivity C-reactive protein (hsCRP) levels of ≥3 mg/L assessed at least 28 days after MI and before first administration of canakinumab. In one embodiment of any use described herein, said patient has high sensitivity C-reactive protein (hsCRP) levels of ≥4 mg/L assessed at least 28 days after MI and before first administration of canakinumab. In one embodiment of any use described herein, said patient has high sensitivity C-reactive protein (hsCRP) levels of ≥5 mg/L assessed at least 28 days after MI and before first administration of canakinumab. In one embodiment of any use described herein, said patient has high sensitivity C-reactive protein (hsCRP) levels of ≥6 mg/L assessed at least 28 days after MI and before first administration of canakinumab. In one embodiment of any use described herein, said patient has high sensitivity C-reactive protein (hsCRP) levels of ≥7 mg/L assessed at least 28 days after MI and before first administration of canakinumab. In one embodiment of any use described herein, said patient has high sensitivity C-reactive protein (hsCRP) levels of ≥8 mg/L assessed at least 28 days after MI and before first administration of canakinumab. In one embodiment of any use described herein, said patient has high sensitivity C-reactive protein (hsCRP) levels of ≥9 mg/L assessed at least 28 days after MI and before first administration of canakinumab. In one embodiment of any use described herein, said patient has high sensitivity C-reactive protein (hsCRP) levels of ≥10 mg/L assessed at least 28 days after MI and before first administration of canakinumab.

In one embodiment of any use of the invention, 150 mg or 300 mg canakinumab is administered. In a preferred embodiment of any use of the invention, 150 mg canakinumab is administered. In a preferred embodiment of any use described herein, canakinumab is administered at the earliest 30 days after MI.

In one embodiment of any use of the invention the reduced level of hsCRP assessed approximately 3 months after first administration of canakinumab is <1.9, <1.8, <1.7, <1.6, <1.5, <1.4, <1.3, <1.2, <1.1, <1.0, <0.9, <0.8, <0.7, <0.6, or <0.5 mg/L. In one embodiment, the reduced level of hsCRP assessed approximately 3 months after first administration of canakinumab is <1.8 mg/L. In another embodiment, the reduced level of hsCRP assessed approximately 3 months after first administration of canakinumab is <1.5 mg/L.

Accordingly, one embodiment of the invention provides canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab approximately every 3 months, and
iv) wherein said patient has a reduced hsCRP level of <2 mg/L assessed approximately 3 months after first administration of canakinumab.

Accordingly, one embodiment provides canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg of canakinumab is administered to the patient at the earliest 30 days after MI, and
iii) wherein said patient will continue to receive about 150 mg canakinumab approximately every 3 months, provided said patient has a reduced hsCRP level of <2 mg/L assessed approximately 3 months after first administration of canakinumab.

Accordingly, one embodiment provides canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg of canakinumab is administered to the patient at the earliest 30 days after MI, and
iii) wherein said patient will continue to receive about 150 mg canakinumab approximately every 3 months, provided said patient has a reduced hsCRP level of <1.8 mg/L assessed approximately 3 months after first administration of canakinumab.

Accordingly, another embodiment provides canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg of canakinumab is administered to the patient at the earliest 30 days after MI, and
iii) wherein said patient will continue to receive about 150 mg canakinumab approximately every 3 months, provided said patient has a reduced hsCRP level of <1.5 mg/L assessed approximately 3 months after first administration of canakinumab.

In another embodiment, provided is the use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab about every 3 months, and
iv) wherein said patient has a reduced hsCRP level of <2 mg/L assessed approximately 3 months after first administration of canakinumab.

In one embodiment, the present invention provides canakinumab for the manufacture of a medicament in reducing the risk of or preventing recurrent cardiovascular (CV) events in a patient that has suffered myocardial infarction (MI), wherein
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab about every 3 months, provided said patient has a reduced hsCRP level of <2 mg/L assessed approximately 3 months after first administration of canakinumab.

Another embodiment provides canakinumab for the manufacture of a medicament in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab about every 3 months, provided said patient has a reduced hsCRP level of <1.8 mg/L assessed approximately 3 months after first administration of canakinumab.

Yet another embodiment provides canakinumab for the manufacture of a medicament in reducing the risk of or preventing recurrent cardiovascular (CV) events in a patient that has suffered myocardial infarction (MI), wherein
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab about every 3 months, provided said patient has a reduced hsCRP level of <1.5 mg/L assessed approximately 3 months after first administration of canakinumab.

In one embodiment, any use of the invention further comprises administering the patient an additional dose of about 300 mg of canakinumab two weeks (+/−3 days) from initial administration of canakinumab.

Accordingly, in one embodiment, the invention provides the use of canakinumab in reducing the risk of or preventing recurrent cardiovascular (CV) events in a patient that has suffered myocardial infarction (MI), wherein
i) said patient has a high sensitivity C-reactive protein (hsCRP) level of ≥2 mg/L assessed at least 28 days after MI and before administration of canakinumab, and wherein
ii) an initial dose of 300 mg of canakinumab is administered to the patient at the earliest 30 days after MI, and wherein
iii) an additional dose of 300 mg of canakinumab is administered two weeks from initial administration and wherein
iii) said patient will continue to receive subsequent doses of 150 mg or preferably 300 mg canakinumab about every 3 months, provided said patient has a reduced hsCRP level of <2 mg/L assessed at least 3 months after initial administration of canakinumab.

In one embodiment of any use provided herein, the patient has reduced hsCRP level of <2 mg/L assessed approximately 3 months and approximately 6 months after first administration of canakinumab.

In another embodiment of any use provided herein, the patient has reduced hsCRP level of <2 mg/L assessed approximately 3 months, approximately 6 months and approximately 9 months after first administration of canakinumab.

In another embodiment of any use provided herein, the patient has reduced hsCRP level of <2 mg/L assessed approximately 3 months, approximately 6 months and approximately 9 months after first administration of canakinumab.

In another embodiment of any use provided herein, the patient has reduced hsCRP level of <2 mg/L assessed approximately 3 months, approximately 6 months, approximately 9 months and approximately 12 months after first administration of canakinumab.

In another embodiment of any use provided herein, the patient has reduced hsCRP level of <2 mg/L first assessed approximately 3 months after first administration of canakinumab and at approximately three-month intervals thereafter.

Accordingly, one embodiment provides canakinumab for use in reducing the risk of or preventing recurrent cardiovascular (CV) events in a patient that has suffered myocardial infarction (MI),
i. wherein said patient has a high sensitivity C-reactive protein (hsCRP) level of ≥2 mg/L assessed at least 28 days after MI and before first administration of canakinumab, and
ii. wherein about 150 mg of canakinumab is administered to the patient at the earliest 30 days after MI, and
iii. wherein said patient will continue to receive about 150 mg canakinumab approximately every 3 months, provided said patient has reduced hsCRP level of <2 mg/L assessed approximately 3 months and approximately 6 months after first administration of canakinumab.

In another embodiment provided is canakinumab for use in reducing the risk of or preventing recurrent cardiovascular (CV) events in a patient that has suffered myocardial infarction (MI),
i. wherein said patient has a high sensitivity C-reactive protein (hsCRP) level of ≥2 mg/L assessed at least 28 days after MI and before first administration of canakinumab, and
ii. wherein about 150 mg of canakinumab is administered to the patient at the earliest 30 days after MI, and
iii. wherein said patient will continue to receive about 150 mg canakinumab approximately every 3 months, provided said patient has reduced hsCRP level of <2 mg/L assessed approximately 3 months, approximately 6 months and approximately 9 months after first administration of canakinumab.

In another embodiment provided is canakinumab for use in reducing the risk of or preventing recurrent cardiovascular (CV) events in a patient that has suffered myocardial infarction (MI),
i. wherein said patient has a high sensitivity C-reactive protein (hsCRP) level of ≥2 mg/L assessed at least 28 days after MI and before first administration of canakinumab, and
ii. wherein about 150 mg of canakinumab is administered to the patient at the earliest 30 days after MI, and
iii. wherein said patient will continue to receive about 150 mg canakinumab approximately every 3 months, provided said patient has reduced hsCRP level of <2 mg/L assessed approximately 3 months, approximately 6 months, approximately 9 months and approximately 12 months after first administration of canakinumab.

In another embodiment provided is canakinumab for use in reducing the risk of or preventing recurrent cardiovascular (CV) events in a patient that has suffered myocardial infarction (MI),
i. wherein said patient has a high sensitivity C-reactive protein (hsCRP) level of ≥2 mg/L assessed at least 28 days after MI and before first administration of canakinumab, and
ii. wherein about 150 mg of canakinumab is administered to the patient at the earliest 30 days after MI, and
iii. wherein said patient will continue to receive about 150 mg canakinumab approximately every 3 months, provided said patient has reduced hsCRP level of <2 mg/L assessed approximately 3 months and approximately 9 months after first administration of canakinumab.

In another embodiment provided is canakinumab for use in reducing the risk of or preventing recurrent cardiovascular (CV) events in a patient that has suffered myocardial infarction (MI),
i. wherein said patient has a high sensitivity C-reactive protein (hsCRP) level of ≥2 mg/L assessed at least 28 days after MI and before first administration of canakinumab, and
ii. wherein about 150 mg of canakinumab is administered to the patient at the earliest 30 days after MI, and
iii. wherein said patient will continue to receive about 150 mg canakinumab approximately every 3 months, provided said patient has reduced hsCRP levels of <2 mg/L first assessed approximately 3 months after first administration of canakinumab and at approximately three-month intervals thereafter.

In one embodiment of any use provided herein, canakinumab is administered at the earliest 30 days after MI.

In one embodiment of any use of the invention, said recurrent CV event is selected from non-fatal MI, non-fatal stroke, cardiovascular (CV) death and hospitalization for unstable angina requiring unplanned revascularization. In another embodiment of any use of the invention, said recurrent CV event is selected from non-fatal MI, non-fatal stroke and cardiovascular (CV) death. In yet another embodiment of any use of the invention said recurrent CV event is non-fatal MI or cardiovascular (CV) death. In another embodiment of any use of the invention said recurrent CV event is non-fatal MI. In another embodiment of any use of the invention said recurrent CV event is hospitalization for unstable angina requiring unplanned revascularization.

In embodiments of any use or method disclosed herein, canakinumab can be administered subcutaneously or intravenously. Canakinumab can be administered in a reconstituted formulation comprising canakinumab at a concentration of 50-200 mg/ml, 50-300 mM sucrose, 10-50 mM histidine, and 0.01-0.1% surfactant and wherein the pH of the formulation is 5.5-7.0. Canakinumab can be administered in a reconstituted formulation comprising canakinumab at a concentration of 50-200 mg/ml, 270 mM sucrose, 30 mM histidine and 0.06% polysorbate 20 or 80, wherein the pH of the formulation is 6.5.

In embodiments of any use or method disclosed herein, canakinumab can also be administered in a liquid formulation comprising canakinumab at a concentration of 50-200 mg/ml, a buffer system selected from the group consisting of citrate, histidine and sodium succinate, a stabilizer selected from the group consisting of sucrose, mannitol, sorbitol, arginine hydrochloride, and a surfactant, e.g., polysorbate 20 or polysorbate 80, and wherein the pH of the formulation is 5.5-7.0. Canakinumab can also be administered in a liquid formulation comprising canakinumab at a concentration of 50-200 mg/ml, 50-300 mM mannitol, 10-50 mM histidine and 0.01-0.1% surfactant, and wherein the pH of the formulation is 5.5-7.0. Canakinumab can also be administered in a liquid formulation comprising canakinumab at a concentration of 50-200 mg/ml, 270 mM mannitol, 20 mM histidine and 0.04% polysorbate 20 or 80, wherein the pH of the formulation is 6.5.

When administered subcutaneously according to any use or method disclosed herein, canakinumab can be administered to the patient in a liquid form contained in a prefilled syringe, autoinjector or as a lyophilized form for reconstitution.

In other embodiments of any method or use of the invention, said patient is concomitantly receiving standard of care treatment reducing the risk of or preventing recurrent CV events. Said standard of care treatment includes but is not limited to lipid lowering agents such as a HMG-CoA reductase inhibitor, e.g., a statin such as lovastatin, pravastatin, simvastatin, fluvastatin, atorvastatin, cerivastatin, mevastatin, pitavastatin, rosuvastatin or mixtures thereof or mixtures with ezetimibe, niacin, amlodipine besylate, inhibitors of proprotein convertase subtilisin/kexin type 9 (PCSK9i) such as alirocumab (Praluent®), evolocumab (Repatha®), bococizumab, inhibitors of cholesterylester transfer protein (CETP) such as anacetrapib, torcetrapib, dalcetrapib, anti-hypertensives such as a calcium channel blocker (e.g., amlodipine, diltiazem, nifedipine, nicardipine, verapamil) or beta-adrenergic blocking drugs such as esmolol, metoprolol, nadolol, penbutolol or anti-hypertensives such as labetalol, metoprolol, hydralazine, nitroglycerin, nicardipine, sodium nitroprusside, clevidipine or a diuretic such as a thiazide diuretic, chlorthalidone, furosemide, hydrochlorothiazide, indapamide, metolazone, amiloride hydrochloride, spironolactone, triamterene, or an angiotensin-converting enzyme (ACE) inhibitor such as ramipril, ramiprilat, captopril, lisinopril or an angiotensin II receptor blocker such as losartan, valsartan, olmesartan, irbesartan, candesartan, telmisartan, eprosartan or an angiotensin receptor-neprilysin inhibitor (ARNI) such as sacubitril/valsartan (Entresto®), or an anticoagulant such as acenocoumarol, coumatetralyl, dicoumarol, ethyl biscoumacetate, phenprocoumon, warfarin heparin, low molecular weight heparin such as bemiparin, certoparin, dalteparin, enoxaparin, nadroparin, parnaparin, reviparin, tinzaparin or an inhibitor of platelet aggregation such clopidogrel, elinogrel, prasugrel, cangrelor, ticagrelor, ticlopidine, cilostazol, dipyridamole, picotamide, abciximab, eptifibatide, tirofiban or terutroban or a Prostaglandin analogue (PGI2) such as beraprost, prostacyclin, iloprost or treprostinil, or COX inhibitors such as aspirin, aloxiprin or carbasalate calcium, indobufen or triflusal or cloricromen or ditazole or 1,3-indandiones such as clorindione, diphenadione or phenindion, or tioclomarol, or direct thrombin (II) inhibitors such as hirudin, bivalirudin, lepirudin, desirudin (bivalent) or argatroban or dabigatran (monovalent) or oligosaccharides such as fondaparinux, idraparinux, or heparinoids such as danaparoid, sulodexide, dermatan sulfate or direct Xa inhibitors xabans such as apixaban, betrixaban, edoxaban, otamixaban, rivaroxaban or REG1 or defibrotide or ramatroban or antithrombin III or protein C (drotrecogin alfa) or fibrinolytics plasminogen activators: r-tPA such as alteplase, reteplase, tenecteplase or UPA such as urokinase or saruplase or streptokinase or anistreplase or monteplase or other serine endopeptidases or ancrod or fibrinolysin; or brinase or citrate or EDTA or oxalate or digitalis, or digoxin, or nesiritide, or oxygen, or a nitrate such as glyceryl trinitrate (GTN)/nitroglycerin, isosorbide dinitrate, isosorbide mononitrate or an analgesic such as morphine sulfate or a renin inhibitor such as aliskiren or an endothelin A receptor inhibitor or an aldosterone inhibitor.

Another embodiment of the invention provides a pharmaceutical composition for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, and wherein said patient has a reduced hsCRP level of <2 mg/L assessed approximately 3 months after first administration of canakinumab.

Another embodiment of any aspect described above include a pharmaceutical composition for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately 3 months after first administration of canakinumab.

In one embodiment, the invention provides the use of high-sensitive C-reactive protein (hsCRP) as a biomarker in identifying a patient for responsiveness to canakinumab for reducing the risk of or preventing recurrent cardiovascular (CV) events in said patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, and wherein said patient has a reduced hsCRP level of <2 mg/L assessed approximately 3 months after first administration of canakinumab.

In another embodiment, the invention provides the use of high-sensitive C-reactive protein (hsCRP) as a biomarker in identifying a patient for responsiveness to canakinumab for reducing the risk of or preventing recurrent cardiovascular (CV) events in said patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately 3 months after the initial administration of canakinumab.

The phrase “identifying a patient” as used herein refers to using the information or data generated relating to the level of hsCRP as referred to herein in a sample of a patient to identify or selecting the patient as more likely to benefit or less likely to benefit from a therapy comprising canakinumab. In one embodiment, a patient is considered to respond to a therapy comprising canakinumab (and, thus, to be more likely to benefit from said therapy), if said therapy reduces the risk of said patient of experiencing a recurrent cardiovascular (CV) event. In one embodiment, said risk is reduced by at least 20%, by at least 21%, by at least 22%, by at least 23%, by at least 24%, by at least 25%, by at least 26%, by at least 27%, by at least 28%, by at least 29% or by at least 30%. Also, a patient is considered not to respond to a therapy comprising canakinumab (and, thus, to be more likely not to benefit from said therapy), if said therapy does not reduce the risk of experiencing a recurrent cardiovascular (CV) event after first administration of canakinumab. In this case, unnecessary health care costs or patient exposure can be avoided, if the medicament is not administered to unresponsive patients.

Another biomarker that is useful is assessing residual inflammatory risk includes downstream mediators of IL-1β such as interleukin-6 (IL-6). IL-6 is a known marker of cardiovascular disease associated with obesity, type 2 diabetes and myocardial infarction. The present inventors also found that administration of canakinumab to stable post-MI patients resulted in lowering of levels of IL-6, a marker for inflammation. Accordingly, in another embodiment of any use according to the invention, IL-6 is used as a biomarker for assessing the response of the stable MI patient to administration of about 150 mg to about 300 mg canakinumab, administered at the earliest 30 days after MI.

### General:

All patents, published patent applications, publications, references and other material referred to herein are incorporated by reference herein in their entirety.

As used herein, the term “comprising” encompasses “including” as well as “consisting,” e.g. a composition “comprising” X may consist exclusively of X or may include something additional, e.g., X+Y.

As used herein, the term “administering” in relation to a compound, e.g., canakinumab or standard of care agent, is used to refer to delivery of that compound by any route of delivery.

As used herein, the term “about” in relation to a numerical value x means, for example, +/−10%.

As used herein, the word “substantially” does not exclude “completely,” e.g., a composition which is “substantially free” from Y may be completely free from Y. Where necessary, the word “substantially” may be omitted from the definition of the disclosure.

As used herein, the term “3 months” includes a time period that extends one week before and one week after the 3 months (3 months+/−1 week). In another embodiment the term “approximately 3 months” includes a time period of 90 days+/−15 days or 90 days+/−10 days.

The term “biomarker”, as used herein, refers generally to a molecule, i.e., a gene (or nucleic acid encoding said gene), protein, the expression of which in a biological sample from a patient can be detected by standard methods in the art, and is predictive or denotes a condition of the patient from which it was obtained. According to the invention, exemplary biomarkers include but are not limited to hsCRP and IL-6.

As used herein, the term “assaying” is used to refer to the act of detecting, identifying, screening, or determining, which act may be performed by any conventional means. For example, a sample may be assayed for the presence of a particular marker by using an ELISA assay, a Northern blot, imaging, etc. to detect whether that marker is present in the sample.

As used herein, the terms “C-reactive protein” and “CRP” refers to serum C-reactive protein, which is used as an indicator of the acute phase response to inflammation. In certain embodiments of the uses and methods described herein, hsCRP levels are assessed in a biological sample, e.g., blood, obtained from the patient. A biological sample from the patient is assayed for the level of hsCRP. As used herein, the term “hsCRP” refers to the level of CRP in the blood as measured by high sensitivity CRP testing. The level of CRP or hsCRP in plasma may be given in any concentration, e.g., mg/dl, mg/L, nmol/L. Levels of CRP or hsCRP may be measured by a variety of well-known methods, e.g., radial immunodiffusion, electroimmunoassay, immunoturbidimetry, ELISA, turbidimetric methods, fluorescence polarization immunoassay, and laser nephelometry. Testing for CRP may employ a standard CRP test or a high sensitivity CRP (hsCRP) test (i.e., a high sensitivity test that is capable of measuring low levels of CRP in a sample, e.g., using laser nephelometry). Kits for detecting levels of CRP or hsCRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, etc.

The term “assaying” is used to mean that a sample may be tested (either directly or indirectly) for either the presence or level of a given marker (e.g., hsCRP or IL-6). It will be understood that, in a situation where the level of a substance denotes a probability, then the level of such substance may be used to guide a therapeutic decision. For example, one may determine the level of hsCRP in a patient by assaying for its presence by quantitative or relatively-quantitative means (e.g., levels relative to the levels in other samples). The disclosed methods involve, inter alia, determining the level of a particular marker, e.g., hsCRP, in a patient.

As used herein, the term “patient” and “subject” are used interchangeably.

As used herein, the term “cardiovascular death” includes sudden cardiac death, death due to acute myocardial infarction (AMI), death due to heart failure, death due to stroke, and death due to other cardiovascular causes.

As used herein, “sudden cardiac death” is a sudden death that occurs in a previously stable patient who does not have a prior terminal condition, such as malignancy not in remission or end-stage chronic lung disease.

Death due to acute myocardial infarction (AMI): refers to a death within 30 days after a myocardial infarction (MI) related to consequences seen immediately after the myocardial infarction, such as progressive congestive heart failure (CHF), inadequate cardiac output, or recalcitrant arrhythmia.

Death due to heart failure or cardiogenic shock refers to death occurring in the context of clinically worsening symptoms and/or signs of heart without evidence of another cause of death and includes sudden death occurring during an admission for worsening heart failure as well as death from progressive heart failure or cardiogenic shock following implantation of a mechanical assist device.

Death due to stroke (intracranial hemorrhage or non-hemorrhagic stroke) refers to death occurring up to 30 days after a suspected stroke based on clinical signs and symptoms as well as neuroimaging and/or autopsy, and where there is no conclusive evidence of another cause of death.

As used herein, “death due to other cardiovascular causes” refers to death due to a cardiovascular cause not included in the above categories (e.g. dysrhythmia, pulmonary embolism, cardiovascular intervention, aortic aneurysm rupture, or peripheral arterial disease). Mortal complications of cardiac surgery or non-surgical revascularization, even if “non-cardiovascular” in nature, should be classified as cardiovascular deaths.

As used herein the term “death of undetermined cause” (presumed cardiovascular) refers to all deaths not attributed to the categories of cardiovascular death or to a non-cardiovascular cause are considered presumed cardiovascular deaths. As used herein, “non-cardiovascular death” is defined as any death not covered by cardiac death or vascular death and is categorized as follows: pulmonary causes, renal causes, gastrointestinal causes, infection (including sepsis), non-infectious causes, malignancy, accident/trauma, suicide, non-cardiovascular system organ failure (e.g. hepatic), hemorrhage, not intracranial or other.

As used herein, the term “myocardial infarction (MI)” refers to “acute myocardial infarction”: the term myocardial infarction (MI) is used when there is evidence of myocardial necrosis in a clinical setting consistent with myocardial ischemia. The term MI includes an ST-elevated MI (STEMI) or a non-ST-elevated MI (NSTEMI). Under these conditions any one of the following criteria meets the diagnosis for MI:

The term “spontaneous MI” refers to the detection of rise and/or fall of cardiac biomarkers with at least one value above the 99^{th} percentile of the upper reference limit (URL) together with evidence of myocardial ischemia with at least one of the following: symptoms of ischemia, ECG changes indicative of new ischemia (ST Elevation−New ST elevation at the J-point in two contiguous leads with the cut-off points: ≥0.2 mV in men or ≥0.15 mV in women in leads V2-V3 and/or ≥0.1 mV in other leads, ST depression and T-wave changes−New horizontal or down-sloping ST depression≥0.05 mV in two contiguous leads; and/or T inversion≥0.1 mV in two contiguous leads with prominent R waves or R/S ratio>1, development of pathological Q waves in the ECG (Any Q-wave in leads V2-V3≥0.02 seconds or QS complex in leads V2 and V3, Q-wave≥0.03 seconds and ≥0.1 mV deep or QS complex in leads I, II, aVL, aVF, or V4-V6 an any two leads of a contiguous lead grouping (I, aVL, V6, V4-V6, II, III, aVF), imaging evidence of new loss of viable myocardium or new regional wall motion abnormality.

The term “percutaneous coronary intervention (PCI) related myocardial infarct” refers to PCI in patients with normal baseline troponin values elevations of cardiac biomarkers above the 99^{th} percentile URL within 24 hours of the procedure are indicative of peri-procedural myocardial necrosis. By convention increases of biomarkers greater than 3×99^{th} percentile URL are consistent with PCI related myocardial infarction. If the cardiac biomarker is elevated prior to PCI a ≥20% increase of the value in that second cardiac biomarker within 24 hours of the PCI and documentation that cardiac biomarkers were decreasing (two samples at least 6 hours apart) prior to the suspected recurrent MI is also consistent with PCI related MI. Symptoms of cardiac ischemia are not required.

The term “CABG related myocardial infarct” refers to CABG in patients with normal baseline troponin, elevations of cardiac biomarkers above 5 times the 99^{th} percentile of the normal reference range during the first 72 hours after CABG, when associated with either new pathological Q waves in at least 2 contiguous leads on the ECG that persist through 30 days or new left bundle branch block (LBBB) or angiographically documented new graft or native coronary artery occlusion or imaging evidence of new loss of viable myocardium.

If the cardiac biomarker is elevated prior to CABG a ≥20% increase of the value in the second cardiac biomarker within 72 hours of CABG AND documentation that the cardiac biomarkers were decreasing (2 samples at least 6 hours apart) prior to the suspected recurrent MI plus either new pathological Q waves in at least 2 contiguous leads on the ECG or new LBBB, angiographically documented new graft or native artery occlusion or imaging evidence or new loss of viable myocardium is consistent with a peri-procedural myocardial infarct after CABG. Symptoms of cardiac ischemia are not required.

Criteria for Prior Myocardial Infarction: Any of the following criteria meets the diagnosis for prior myocardial infarction: development of new pathological Q waves with or without symptoms, imaging evidence of a region of loss of viable myocardium that is thinned and fails to contract in the absence of a non-ischemic cause, pathological findings of a healed or healing myocardial infarction

ECG changes associated with prior Myocardial Infarction:
Any Q wave in leads V2-V3≥0.02 seconds or QS complex in leads V2 and V3
Q-wave≥0.03 seconds and ≥0.1 mV deep or QS complex in leads I, II, aVL, aVF, or V4-V6 in any two leads of a contiguous lead grouping (I, aVL, V6, V4-V6, II, III, and aVF)
R-wave≥0.04 seconds in V1-V2 and R/S≥1 with a concordant positive T-wave in the absence of a conduction defect

Criterion for Reinfarction: In patients where recurrent MI is suspected from clinical signs or symptoms following the initial infarction, an immediate measurement of the employed cardiac biomarker is recommended. A second sample should be obtained 3-6 hours later. Recurrent infarction is diagnosed if there is a ≥20% increase of the value in the second sample. This value should exceed the 99^{th} percentile URL. However if cardiac biomarkers are elevated prior to the suspected new MI, there must also be documentation of decreasing values (two samples at least 6 hours apart) prior to the suspected new MI. If the values are falling criteria for reinfarction by further measurement of biomarkers together with features of the ECG or imaging can be applied.

ECG diagnosis of reinfarction following the initial infarction: may be confounded by the initial evolutionary ECG changes. Reinfarction should be considered when the ST elevation≥0.1 mV reoccurs in an inpatient having a lesser degree of ST elevation or new pathognomonic Q-waves, in at least two contiguous leads, particularly when associated with ischemic symptoms for 10 minutes or longer. The re-evaluation of the ST segment can, however also be seen in threatening myocardial rupture and should lead to additional diagnostic work-up. ST depression or LBBB on their own should not be considered valid criteria for Myocardial Infarction.

If biomarkers are increasing or peak is not reached then there is insufficient data to diagnose recurrent MI.

Clinical Classification of different types of Myocardial Infarction:
Type 1—Spontaneous MI related to ischemia due to a primary coronary event such as plaque erosion and/or rupture, fissuring or dissection.
Type 2—MI secondary to ischemia due to either increased oxygen demand or decreased supply, e.g. coronary artery spasm, anemia, hypotension, coronary embolism, arrhythmias, hypertension or hypotension.
Type 3—Sudden unexpected cardiac death including cardiac arrest, often with symptoms suggestive of myocardial ischemia accompanied by presumably new ST elevation, or new LBBB, or evidence of fresh thrombus in a coronary artery by angiography and/or at autopsy, but death occurring before blood samples could be obtained or at a time before the appearance of cardiac biomarkers in the blood.
Type 4a—MI associated with PCI (Percutaneous Coronary Intervention).
Type 4b—MI associated with stent thrombosis as documented by autopsy or angiography.
Type 5—MI associated with CABG (Coronary artery bypass grafting)

The term “silent MI”: the following criteria will be used by the central ECG reading vendor to define interval “silent” (no clinical symptoms or signs) MI between baseline and yearly ECGs (Surawicz B et al, Chou's electrocardiography in clinical practice: adult and pediatric. Philadelphia: Saunders; 2001):

Myocardial infarctions are reported only on the basis of pathologic Q waves. Pathologic Q waves are defined as Q wave duration>40 ms and Q/R ratio=1/3.

Any Q wave in V1 or V2 that is followed by an R wave should be considered abnormal.

When pathologic Q waves (i.e., myocardial infarction) are present, ST elevation or T wave inversion may be used to classify the infraction as New or Acute. However, ST elevation or T wave inversion in the absence of pathologic Q waves are not sufficient criteria for diagnosis of myocardial infarction.
Anterolateral MI—Pathologic Q waves in leads V3-V6.
Anterior MI—Pathologic Q waves in V3 and V4.
Anteroseptal MI—Pathologic Q waves or QS in leads V1-V4.
Extensive Anterior MI—Pathologic Q waves in leads I, aVL, and V1-V6.
High lateral MI—Pathologic Q waves in leads I and aVL.
Inferior MI—Pathologic Q waves or QS in at least two of the inferior leads: aVF, III, II.
Lateral MI—Pathologic Q waves in leads I, aVL, and V5-V6.
Septal MI—Pathologic Q waves or QS in leads V1-V2, (V3). In the presence of LAHB or LVH a Q or QS in V3 is required.
Posterior MI—Initial R wave duration 40 ms in V1 or V2, and R>S and upright T wave; Inferior or Lateral MI are usually also present.
   The term “new MI” as used herein is based on criteria for MI more stringent than the Expert Consensus Document criteria, requiring Q waves to be ≥0.04 sec in duration and an R/S ratio≥1/3. These criteria (drawn from the cardiology literature) are designed to minimize the false positive detection of MIs due to very small physiologic Q waves in the inferior and anterolateral leads.

As used herein, the term “stroke” is defined as the rapid onset of a new persistent neurological deficit attributed to an obstruction in cerebral blood flow and/or cerebral hemorrhage with no apparent non-vascular cause (e.g. tumor, trauma, infection). Available neuroimaging studies will be considered to support the clinical impression and to determine if there is a demonstrable lesion compatible with an acute stroke. Non-fatal strokes will be classified as ischemic, hemorrhagic or unknown.

As used herein the term “unstable angina requiring unplanned revascularization” is defined as no elevation in cardiac biomarkers and clinical presentation (one of the following) with cardiac symptoms lasting ≥10 minutes and considered to be myocardial ischemia on final diagnosis (rest angina or new onset (<2 months) severe angina (CCS classification severity≥III; Grading of Angina Pectoris According to Canadian Cardiovascular Society Classification) or increasing angina (in intensity, duration and/or frequency) and severe recurrent ischemia requiring urgent revascularization: as defined by an episode of angina prompting the performance of coronary revascularization on the index hospitalization or an episode of recurrent angina after discharge that resulted in re-hospitalization during which coronary revascularization was performed; and at least one of the following: new or worsening ST or T segment changes on ECG, ST Elevation (new ST elevation at the J point in two anatomically contiguous leads with the cut-off points: ≥0.2 mV in men (>0.25 mV in men<40 years) or ≥0.15 mV in women in leads V2-V3 and/or ≥0.1 mV in other leads), ST depression and T-wave Evidence of ischemia on stress testing with cardiac imaging, evidence of ischemia on stress testing without cardiac imaging but with angiographic evidence of ≥70% lesion, and/or thrombus in the epicardial coronary artery or initiation/increased dosing of anti-anginal therapy, angiographic evidence of ≥70% lesion and/or thrombus in an epicardial coronary artery.

As used herein “coronary revascularization” is defined as an invasive procedure, which usually follows coronary angiography, wherein either percutaneous transluminal intervention, followed by Stent Placement, Balloon Angioplasty, or CABG is performed to relieve obstructed coronary arteries. A team of medical professionals lead by either an invasive cardiologist (percutaneous transluminal intervention, followed by stent placement, balloon angioplasty) or a thoracic surgeon (CABG), who performs the described procedures.

As used herein the term “non-coronary revascularization” is defined as vascular surgery or percutaneous intervention. Vascular surgery is defined as the placement of a conduit with or without proximal and/or distal anastamoses. Percutaneous intervention is defined as balloon inflation with or without stenting.

As used herein, the term “atherosclerosis” occurs when fatty material and a substance called plaque builds up on the walls of the arteries. This causes their lumen to get narrow.

As used herein, the term “MACE” comprises non-fatal heart attack, non-fatal stroke and cardiovascular (CV) death.

It is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

Other features, objects, and advantages of the invention will be apparent from the description and drawings, and from the claims.

The following Examples illustrate the invention described above; they are not, however, intended to limit the scope of the invention in any way.

### Example

The Example below is set forth to aid in the understanding of the invention but is not intended, and should not be construed, to limit its scope in any way.

A Randomized, Double-Blind, Placebo-Controlled, Event-Driven Trial of Quarterly Subcutaneous Canakinumab in the Prevention of Recurrent Cardiovascular Events Among Stable Post-Myocardial Infarction Patients with Elevated hsCRP.

This study was designed as a multi-center, randomized, parallel group, placebo-controlled, double-blind, event-driven trial to provide definitive evidence on the effects of canakinumab on cardiovascular adverse events in patients with recent MI and elevated inflammatory burden as evidenced by elevated hsCRP. This study design was the most robust clinical trial design to test the hypothesis that anti-inflammatory treatment with canakinumab reduce major adverse cardiovascular events.

### Rationale of Study Design

### Trial Population.

Patients were eligible for enrollment if they had a prior history of myocardial infarction and had blood levels of hsCRP of 2 mg/L or greater despite use of aggressive secondary prevention strategies. The trial excluded from enrollment those with a history of chronic or recurrent infection, prior malignancy other than basal cell skin carcinoma, suspected or known immunocompromised state, a history of or high risk for tuberculosis or HIV-related disease, or ongoing use of other systemic anti-inflammatory treatments.

### Inclusion Criteria

Patients eligible for inclusion in the study had to fulfill all of the following criteria:
1. Written informed consent obtained before any assessment performed.
2. Male, or Female of non-child-bearing potential
3. Age≥18 years at Visit 1.
4. Documented spontaneous MI (diagnosed according to the universal MI criteria with or without evidence of ST segment elevation) at least 30 days before randomization (Duewell P et al, Nature. 2010; 464(7293): 1357-61).

Diagnosis of the qualifying MI should be based on medical history of clinical symptoms consistent with myocardial ischemia associated with elevation of cardiac biomarkers above the 99th percentile of the upper reference limit (preferably troponin) OR development of new pathological Q waves regardless of symptoms. For details, refer to the Universal Definition of MI (Duewell P et al, Nature. 2010; 464(7293):1357-61).

a. Acute MI (hospitalization records): requires documentation of a rise and/or fall of cardiac biomarkers (preferably troponin) with at least one value above the 99th percentile of the upper reference limit (URL) or above criteria diagnostic for MI and evidence of myocardial ischemia as demonstrated by at least one of the following:
i. Symptoms of ischemia
ii. ECG changes indicative of new ischemia (new ST-T changes or new LBBB)
iii. Development of pathologic Q waves
iv. Imaging evidence of new loss of viable myocardium or new regional wall motion abnormality
b. Prior MI (no hospital records for acute event available): requires documentation of any one of the following:
i. Development of pathological Q waves, with or without symptoms
ii. Imaging evidence of a region of loss of viable myocardium that is thinned and fails to contract, in the absence of a non-ischemic cause
iii. Pathologic findings of a healed or healing MI
Patients with MI resulting from PCI or CABG were not eligible
5. Have an hsCRP≥2 mg/L (collected less than 60 days prior to Visit 2 and performed at the central laboratory, which is a minimum of 28 days after qualifying MI or after any PCI performed separately from qualifying MI) on stable (at least 4 weeks) long term (cardiovascular) medications (standard of care).

Randomization.

Patients were initially randomized to canakinumab 150 mg, canakinumab 300 mg, or placebo in a 1:1:1 ratio. After the enrollment of 741 participants, a 50 mg dose was added at regulatory request, with the randomization ratio adjusted accordingly; we sought to achieve a final randomization ratio of 1.5:1:1:1. All study-drug doses and placebo were administered subcutaneously once every three months; for the 300 mg dose, the regimen was 300 mg every two weeks for the first two doses, then once every three months. Randomization was performed with the use of a centralized computer system, with stratification by time since index myocardial infarction and by trial part (before versus after inclusion of the 50 mg dose).

### End Points.

The primary efficacy end point was time to first occurrence of nonfatal myocardial infarction, any nonfatal stroke, or cardiovascular death. The trial had two key secondary efficacy end points. The first key secondary end point included the components of the primary end point as well as hospitalization for unstable angina requiring urgent revascularization. The two other pre-specified secondary end points were all-cause mortality and the composite of nonfatal myocardial infarction, any nonfatal stroke, or all-cause mortality. All components of these end points were adjudicated by an end point adjudication committee, with members masked to study-drug assignment.

### Statistical Analysis.

Distributions of percent change from baseline in hsCRP and lipid levels were compared between placebo and each canakinumab group at intervals up to 48 months. Similar comparisons were made for IL-6 up to 12 months. Log-rank tests and Cox proportional-hazards models, stratified by time since index myocardial infarction and trial part, were used to analyze the pre-specified primary and key secondary cardiovascular outcomes that occurred during trial follow-up according to the intention-to-treat principle. Formal evaluation of significance for individual doses, adjusted for multiplicity, followed a closed testing procedure. Based on the closed testing procedure, and using the pre-specified allocation of alpha error, the two-sided P value thresholds for statistical significance for the primary end point were 0.01058 for the test of the 300 mg dose of canakinumab versus placebo and 0.02115 for the tests of the other two doses versus placebo. The closed testing procedure also specified that formal significance testing for the key secondary end points would be performed for any given dose only if the significance threshold for the primary end point for that dose had been met.

While the primary analysis strategy was based on pair-wise comparisons of individual dose groups to the placebo group, comparisons were also made between incidence rates on placebo and incidence rates across ascending canakinumab doses (using scores of 0, 1, 3, and 6 proportional to doses in a trend analysis) and for the combined active canakinumab treatment groups versus placebo. In addition, on-treatment analyses were performed with follow-up for each patient censored 119 days after the last study injection received. The significance thresholds for these tests were not adjusted for multiplicity. Similar analyses were used for adverse events. All P values are two-sided and all confidence intervals computed at the 95% level.

### Patients.

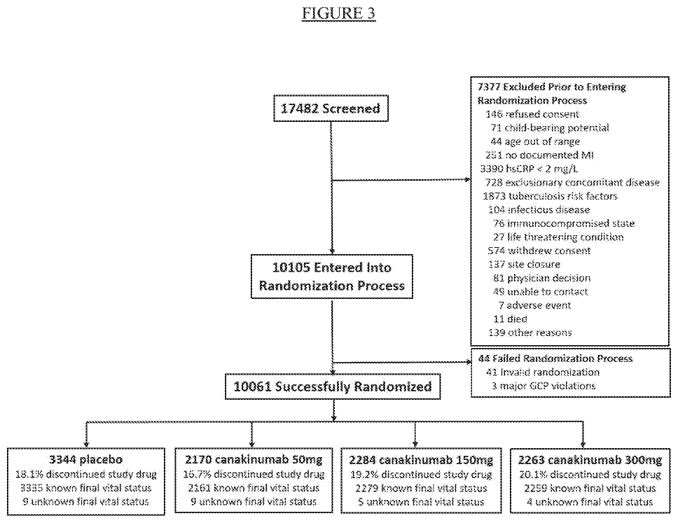

Trial enrollment began in April 2011 and was completed in March 2014; the last trial visit was in June 2017. Of 17,482 post-infarction patients who underwent screening in the central laboratory, 10,061 (57.6%) were correctly randomized and received at least one dose of trial medication (FIG. 3). The most common reasons for exclusion were hsCRP less than 2 mg/L (46% of excluded subjects), active tuberculosis or tuberculosis risk factors (25.4%), and exclusionary concomitant disorders (9.9%).

The mean age of randomized participants was 61 years, 26% were women, and 40% had diabetes (Table 1). Most participants had undergone prior revascularization procedures (67% percutaneous coronary interventions, 14% coronary bypass surgery). At baseline, anti-thrombotic therapy was taken by 95%, lipid-lowering therapy by 93%, anti-ischemia agents by 91%, and inhibitors of the renin-angiotensin system by 79%. The median hsCRP at entry was 4.2 mg/L and the median LDL cholesterol was 82 mg/dL.

TABLE 1
Characteristics of trial participants

| | Canakinumab Dose (SC q 3 months) | | | | |
|---|---|---|---|---|---|
| | Placebo | 50 mg | 150 mg | 300 mg | All Doses |
| Characteristic | (N = 3344) | (N = 2170) | (N = 2284) | (N = 2263) | (N = 6717) |
| Age (yr), Mean (SD) | 61.1 (10.0) | 61.1 (10.1) | 61.2 (10.0) | 61.1 (10.1) | 61.1 (10.1) |
| Female sex, N (%) | 865 (25.9) | 541 (24.9) | 575 (25.2) | 606 (26.8) | 1722 (25.6) |
| Current smoking, N | 765 (22.9) | 531 (24.5) | 534 (23.4) | 536 (23.7) | 1601 (23.8) |
| (%) | | | | | |
| Body mass index | 29.7 (26.6, | 29.9 (26.6, | 29.8 (26.5, | 29.8 | 29.9 |
| (kg/m²)† | 33.8) | 33.9) | 33.7) | (26.5, | (26.6, |
| | | | | 33.8) | 33.8) |
| Hypertension, N (%) | 2644 (79.1) | 1751 | 1814 | 1799 | 5364 |
| | | (80.7) | (79.4) | (79.5) | (79.9) |
| Diabetes, N (%) | 1333 (39.9) | 854 (39.4) | 954 (41.8) | 888 (39.2) | 2696 |
| | | | | | (40.1) |
| Qualifying myocardial | | | | | |
| infarction, N (%) | | | | | |
| STEMI | 1807 (54.0) | 1231 | 1231 | 1213 | 3675 |
| | | (56.7) | (53.9) | (53.6) | (54.7) |
| Non-STEMI | 1132 (33.9) | 710 (32.7) | 781 (34.2) | 761 (33.6) | 2252 |
| | | | | | (33.5) |
| Unknown/missing | 405 (12.1) | 229 (10.6) | 272 (11.9) | 289 (12.8) | 790 (11.8) |
| History of PCI, N (%) | 2192 (65.6) | 1454 | 1555 (68.1)* | 1509 | 4518 |
| | | (67.0) | | (66.7) | (67.3) |
| History of CABG, N | 469 (14.0) | 302 (13.9) | 324 (14.2) | 316 (14.0) | 942 (14.0) |
| (%) | | | | | |
| History of congestive | 721 (21.6) | 451 (20.8) | 478 (20.9) | 523 (23.1) | 1452 |
| heart failure, N | | | | | (21.6) |
| (%) | | | | | |
| Lipid lowering | 3132 (93.7) | 2038 (94.0) | 2114 (92.7) | 2113 (93.5) | 6265 (93.3) |
| therapy, N (%) | | | | | |
| Statin, N (%) | 3045 (91.1) | 1990 | 2065 | 2057 | 6112 |
| | | (91.7) | (90.6) | (91.1) | (91.0) |
| Renin-angiotensin | 2665 (79.8) | 1718 (79.3) | 1817 (79.8) | 1792 (79.6) | 5327 (79.3) |
| inhibitors, N (%) | | | | | |
| Anti-ischemia | 3080 (92.1) | 1974 (91.0) | 2079 (91.2) | 2058 (91.1) | 6111 (91.0) |
| agents,** N (%) | | | | | |
| Antithrombotics/ | 3188 (95.3) | 2059 (94.9) | 2157 (94.6) | 2149 (95.1) | 6365 (94.8) |
| Anti-coagulants, | | | | | |
| N (%) | | | | | |
| hsCRP (mg/L)† | 4.1 (2.75, | 4.25 (2.80, | 4.25 (2.85, | 4.15 | 4.2 (2.80, |
| | 6.85) | 7.15) | 7.05) | (2.85, | 7.10) |
| | | | | 7.15) | |
| IL-6 (ng/L)† | 2.61 (1.80, | 2.53 (1.80, | 2.56 (1.74, | 2.59 | 2.56 |
| | 4.06) | 4.17) | 4.11) | (1.79, | (1.77, |
| | | | | 4.08) | 4.13) |
| Total cholesterol | 161 (137, 190) | 159 (136, | 159 (136, | 161 (137, | 160 (136, |
| (mg/dL)† | | 189) | 188) | 189) | 189) |
| LDL cholesterol | 82.8 (64.2, | 81.2 (62.3, | 82.4 (63.4, | 83.5 | 82.0 |
| (mg/dL)† | 107.5) | 106.0) | 106.0) | (64.0, | (63.0, |
| | | | | 108.0) | 106.7) |
| HDL cholesterol | 44.5 (37.1, | 43.7 (37.0, | 43.7 (36.3, | 44.0 | 43.7 |
| (mg/dL)† | 52.6) | 52.2) | 52.0)* | (36.7, | (36.7, |
| | | | | 53.0) | 52.2)* |
| Triglycerides | 139 (100, 194) | 140 (102, | 139 (101, | 138 (103, | 139 (102, |
| (mg/dL)† | | 198) | 196) | 194) | 196) |
| eGFR (mL/min/1.73 | 79.0 (65.0, | 79.0 (64.0, | 79.0 (64.5, | 78.0 | 78.5 |
| m²)† | 93.0) | 92.0) | 93.0) | (64.0, | (64.0, |
| | | | | 93.0) | 93.0) |
| Loss to follow-up N, | 9 (0.27) | 9 (0.41) | 5 (0.22) | 4 (0.18) | 18 (0.27) |
| (%) | | | | | |

SC = subcutaneously; SD = standard deviation; STEMI = ST elevation myocardial infarction; PCI = percutaneous coronary intervention; CABG = coronary bypass graft surgery; hsCRP = high sensitivity C-reactive protein; IL-6 = interleukin 6; HDL = high density lipoprotein cholesterol; LDL = low density lipoprotein cholesterol; eGFR = estimated glomerular filtration rate
*P-value < 0.05 in comparison of canakinumab to placebo.
**Beta-blocking agents, nitrates, or calcium channel blocking agents
†Median (IQR) values are presented for all measured plasma variables and body mass index

### Effects on Inflammatory Biomarkers and Lipid Levels.

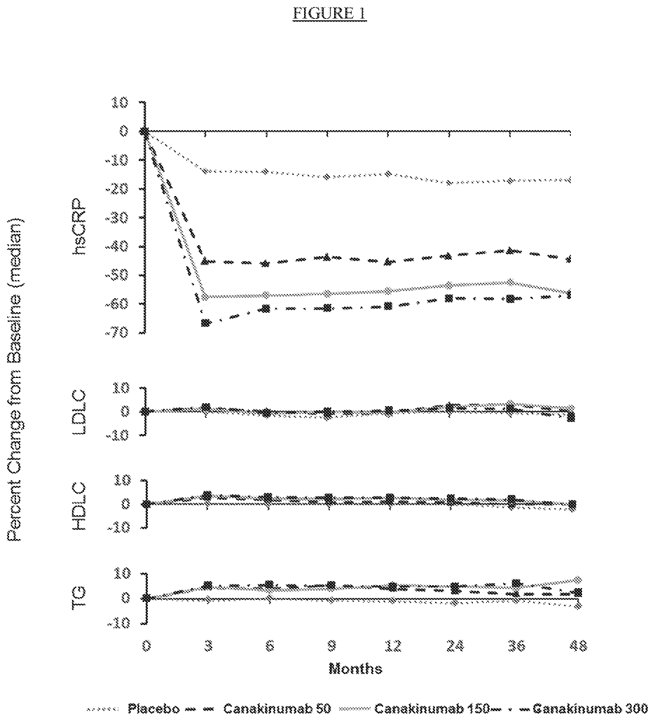

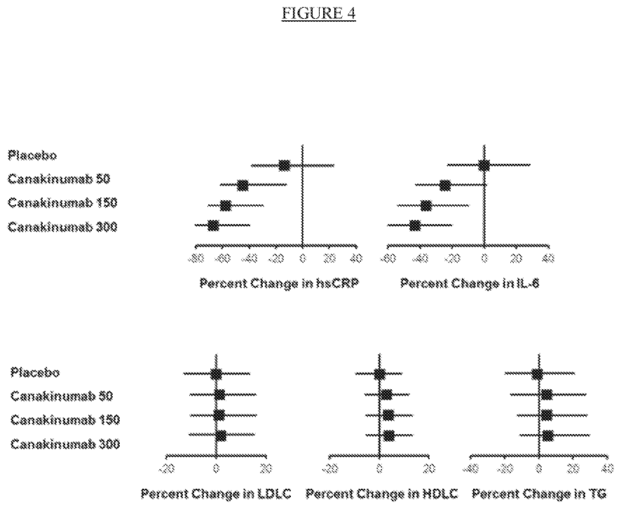

Compared to placebo, at 48 months, hsCRP was reduced by 26%, 37%, and 41% in the canakinumab 50 mg, 150 mg, and 300 mg groups, respectively (all P-values<0.001 in comparisons of the median percent change on canakinumab to the median percent change on placebo) (FIG. 1, FIG. 4, and Tables 2-6). Similar effects were observed for IL-6 (measured up to 12 months). By contrast, canakinumab use resulted in no reduction in LDL cholesterol or HDL cholesterol, and a 4 to 5% median increase in triglycerides.

TABLE 2
Effects of 3-month treatment with canakinumab on hsCRP, IL-6, and lipid levels.
P-values reflect change from baseline.

| | Canakinumab Dose (SC q 3 months) | | | | |
|---|---|---|---|---|---|
| Biomarker | Placebo | 50 mg | 150 mg | 300 mg | All Doses |
| hsCRP (mg/L) Baseline median | 4.05 | 4.05 | 4.15 | 4.05 | 4.10 |
| 3-month median | 3.50 | 2.20 | 1.8 | 1.30 | 1.80 |
| Change median, %) | −13.8 | −45.1 | −57.6 | −66.7 | −56.8 |
| P-value | | <0.001 | <0.001 | <0.001 | <0.001 |
| IL-6 (ng/L) Baseline median | 2.58 | 2.53 | 2.54 | 2.56 | 2.55 |
| 3-month median | 2.60 | 1.98 | 1.64 | 1.43 | 1.64 |
| Change median, % | 0.00 | −24.7 | −36.4 | −43.3 | −35.3 |
| P-value | | <0.001 | <0.001 | <0.001 | <0.001 |
| LDLC (mg/dL) Baseline | 82.8 | 81.2 | 82.0 | 83.1 | 82.0 |
| 3-month median | 82.0 | 82.4 | 84.0 | 83.1 | 83.1 |
| Change median, % | 0.00 | 1.4 | 1.1 | 1.9 | 1.5 |
| P-value | | 0.002 | 0.015 | 0.001 | <0.001 |
| HDLC (mg/dL) Baseline median | 44.5 | 43.7 | 43.7 | 44.0 | 44.0 |
| 3-month median | 44.9 | 44.9 | 45.2 | 46.0 | 45.2 |
| Change median, % | 0.00 | 2.9 | 3.7 | 3.8 | 3.5 |
| P-value | | <0.001 | <0.001 | <0.001 | <0.001 |
| TG (mg/dL) Baseline median | 138.9 | 140.7 | 138.1 | 137.5 | 138.9 |
| 3-month median | 138.9 | 148.7 | 143.9 | 145.1 | 146.0 |
| Change median, % | −0.7 | 4.5 | 4.7 | 5.4 | 4.8 |
| P-value | | <0.001 | <0.001 | <0.001 | <0.001 |

LDLC = low density lipoprotein (LDL) cholesterol, HDLC = high density lipoprotein (HDL) cholesterol, TG = triglycerides, IL-6 = interleukin-6, SC = subcutaneous, q = quarterly

TABLE 3
Effects of 12-month treatment with canakinumab on hsCRP, IL-6, and lipid levels.
P-values reflect change from baseline.

| | Canakinumab Dose (SC q 3 months) | | | | |
|---|---|---|---|---|---|
| Biomarker | Placebo | 50 mg | 150 mg | 300 mg | All Doses |
| hsCRP (mg/L) Baseline median | 4.00 | 4.10 | 4.05 | 4.05 | 4.05 |
| 12-month median | 3.40 | 2.30 | 1.80 | 1.50 | 1.80 |
| Change median, % | −14.6 | −45.3 | −55.6 | −61.0 | −54.5 |
| P-value | | <0.001 | <0.001 | <0.001 | <0.001 |
| IL-6 (ng/L) Baseline median | 2.57 | 2.52 | 2.50 | 2.52 | 2.52 |
| 12-month median | 2.63 | 2.03 | 1.71 | 1.60 | 1.75 |
| Change median, % | 3.52 | −19.5 | −33.8 | −37.7 | −31.1 |
| P-value | | <0.001 | <0.001 | <0.001 | <0.001 |
| LDLC (mg/dL) Baseline | 83.0 | 80.4 | 82.0 | 83.1 | 82.0 |
| 12-month median | 82.4 | 84.0 | 82.4 | 84.0 | 83.5 |
| Change median, % | 0.00 | 1.53 | 0.81 | 0.00 | 0.88 |
| P-value | | 0.04 | 0.22 | 0.64 | 0.11 |
| HDLC (mg/dL) Baseline median | 44.1 | 44.0 | 43.8 | 44.0 | 44.0 |
| 12-month median | 44.5 | 44.5 | 44.9 | 45.0 | 44.9 |
| Change median, % | 0.00 | 1.06 | 2.67 | 2.75 | 2.22 |
| P-value | | <0.001 | <0.001 | <0.001 | <0.001 |
| TG (mg/dL) Baseline median | 138.9 | 139.8 | 138.9 | 138.1 | 138.9 |
| 12-month median | 137.2 | 146.9 | 144.6 | 147.8 | 146.9 |
| Change median, % | −1.0 | 4.0 | 5.7 | 4.7 | 4.7 |
| P-value | | <0.001 | <0.001 | <0.001 | <0.001 |

LDLC = low density lipoprotein (LDL) cholesterol, HDLC = high density lipoprotein (HDL) cholesterol, TG = triglycerides, IL-6 = interleukin-6, SC = subcutaneous, q = quarterly

TABLE 4
Effects of 24-month treatment with can akinumab on hsCRP and
lipid levels. P-values reflect change from baseline.

| | Canakinumab Dose (SC q 3 months) | | | | |
|---|---|---|---|---|---|
| Biomarker | Placebo | 50 mg | 150 mg | 300 mg | All Doses |
| hsCRP (mg/L) Baseline median | 3.95 | 4.00 | 4.00 | 4.00 | 4.00 |
| 24-month median | 3.40 | 2.30 | 1.80 | 1.60 | 1.90 |
| Change median, % | −17.9 | −43.2 | −53.6 | −58.1 | −52.5 |
| P-value | | <0.001 | <0.001 | <0.001 | <0.001 |
| LDLC (mg/dL) Baseline | 83.0 | 80.8 | 82.0 | 82.4 | 82.0 |
| median | | | | | |
| 24-month median | 83.0 | 84.0 | 84.3 | 83.0 | 85.9 |
| Change median, % | 0.0 | 2.2 | 2.1 | 1.5 | 1.9 |
| P-value | | 0.014 | 0.14 | 0.30 | 0.01 |
| HDLC (mg/dL) Baseline median | 44.1 | 44.0 | 44.0 | 44.0 | 44.0 |
| 24-month median | 44.0 | 44.1 | 44.9 | 45.2 | 44.9 |
| Change median, % | 0.00 | 0.76 | 1.92 | 2.45 | 1.90 |
| P-value | | 0.07 | <0.001 | <0.001 | <0.001 |
| TG (mg/dL) Baseline median | 138.9 | 139.8 | 138.9 | 138.1 | 138.9 |
| 24-month median | 135.9 | 142.5 | 146.0 | 145.9 | 145.1 |
| Change median, % | −2.0 | 2.9 | 5.3 | 4.9 | 4.6 |
| P-value | | <0.001 | <0.001 | <0.001 | <0.001 |

LDLC = low density lipoprotein (LDL) cholesterol, HDLC = high density lipoprotein (HDL) cholesterol, TG = triglycerides, SC = subcutaneous, q = quarterly

TABLE 5
Effects of 36-month treatment with can akinumab on hsCRP and lipid levels. P-
values reflect change from baseline.

| | Canakinumab Dose (SC q 3 months) | | | | |
|---|---|---|---|---|---|
| Biomarker | Placebo | 50 mg | 150 mg | 300 mg | All Doses |
| hsCRP (mg/L) Baseline median | 4.05 | 4.10 | 4.10 | 4.10 | 4.10 |
| 36-month median | 3.50 | 2.40 | 2.00 | 1.70 | 2.00 |
| Change median, % | −17.1 | −41.4 | −52.6 | −58.3 | −51.5 |
| P-value | | <.001 | <0.001 | <0.001 | <0.001 |
| LDLC (mg/dL) Baseline | 83.1 | 80.8 | 82.0 | 82.4 | 81.6 |
| median | | | | | |
| 36-month median | 82.8 | 83.1 | 84.0 | 83.5 | 83.9 |
| Change median, % | −0.5 | 3.1 | 3.3 | 1.2 | 2.5 |
| P-value | | 0.002 | <0.001 | 0.04 | <0.001 |
| HDLC (mg/dL) Baseline median | 44.1 | 44.0 | 43.7 | 44.0 | 44.0 |
| 36-month median | 44.0 | 44.0 | 44.5 | 45.0 | 44.5 |
| Change median, % | −1.4 | 0.00 | 1.6 | 2.1 | 1.3 |
| P-value | | 0.01 | <0.001 | <0.001 | <0.001 |
| TG (mg/dL) Baseline median | 138.9 | 140.7 | 139.8 | 138.1 | 139.4 |
| 36-month median | 134.5 | 142.2 | 146.9 | 144.2 | 144.9 |
| Change median, % | −0.6 | 2.4 | 4.5 | 6.2 | 3.9 |
| P-value | | 0.01 | <0.001 | <0.001 | <0.001 |

LDLC = low density lipoprotein (LDL) cholesterol, HDLC = high density lipoprotein (HDL) cholesterol, TG = triglycerides, SC = subcutaneous, q = quarterly

TABLE 6
Effects of 48-month treatment with can akinumab on hsCRP
and lipid levels. P-values reflect change from baseline.

| | Canakinumab Dose (SC q 3 months) | | | | |
|---|---|---|---|---|---|
| Biomarker | Placebo | 50 mg | 150 mg | 300 mg | All Doses |
| hsCRP (mg/L) Baseline median | 4.15 | 4.20 | 4.35 | 4.30 | 4.30 |
| 48-month median | 3.60 | 2.50 | 1.90 | 1.90 | 2.10 |
| Change median, % | −17.0 | −44.4 | −56.3 | −57.1 | −52.9 |
| P-value | | <.001 | <0.001 | <0.001 | <0.001 |
| LDLC (mg/dL) Baseline | 85.1 | 84.7 | 82.4 | 86.0 | 84.7 |
| median | | | | | |
| 48-month median | 82.4 | 84.0 | 84.7 | 85.0 | 84.3 |
| Change median, % | −1.4 | 0.0 | 1.69 | −2.8 | 0.0 |
| P-value | | 0.19 | 0.004 | 0.67 | 0.045 |
| HDLC (mg/dL) Baseline median | 44.1 | 44.5 | 43.7 | 44.1 | 44.1 |
| 48-month median | 43.7 | 44.5 | 44.0 | 44.9 | 44.1 |
| Change median, % | −2.25 | 0.00 | 0.00 | 0.00 | 0.00 |
| P-value | | 0.007 | <0.001 | <0.001 | <0.001 |
| TG (mg/dL) Baseline median | 141.6 | 138.0 | 140.7 | 138.1 | 138.9 |
| 48-month median | 138.9 | 139.8 | 152.2 | 138.9 | 144.2 |
| Change median, % | −3.2 | 1.7 | 7.5 | 2.3 | 3.8 |
| P-value | | 0.026 | <0.001 | 0.020 | <0.001 |

LDLC = low density lipoprotein (LDL) cholesterol, HDLC = high density lipoprotein (HDL) cholesterol, TG = triglycerides, SC = subcutaneous, q = quarterly

### Follow-Up and Effects on Clinical End Points.

By the end of follow-up, 18.1% of patients in the placebo group had discontinued study drug, as compared to 18.7% of patients in the combined canakinumab groups (FIG. 3). At a median follow-up of 3.7 years, the incidence rates for the primary end point (which included nonfatal myocardial infarction, nonfatal stroke, or cardiovascular death) in the placebo, 50 mg, 150 mg, and 300 mg groups were 4.50, 4.11, 3.86, and 3.90 per 100 person-years, respectively (Table 7).

TABLE 7
Incidence rates (per 100 person years) and hazard ratios for major clinical outcomes
and all-cause mortality

| | | | | | | P-value |
|---|---|---|---|---|---|---|
| | | Canakinumab Dose (SC q 3 months) | | | | for |
| | | | | | All | trend |
| | Placebo | 50 mg | 150 mg | 300 mg | Doses | across |
| Clinical Outcome | (N = 3344) | (N = 2170) | (N = 2284) | (N = 2263) | (N = 6717) | doses |
| Primary end point* | | | | | | |
| Incidence rate, (N) | 4.50 | 4.11 | 3.86 | 3.90 | 3.95 | 0.020 |
| | (535) | (313) | (320) | (322) | (955) | |
| Hazard ratio | 1.00 | 0.93 | 0.85 | 0.86 | 0.88 | |
| 95% CI | (referent) | 0.80-1.07 | 0.74-0.98 | 0.75-0.99 | 0.79-0.97 | |
| P | (referent) | 0.30† | 0.021^{#} | 0.031† | 0.015 | |
| Key secondary | | | | | | |
| cardiovascular | | | | | | |
| end point** | | | | | | |
| Incidence rate, (N) | 5.13 | 4.56 | 4.29 | 4.25 | 4.36 | 0.003 |
| | (601) | (344) | (352) | (348) | (1044) | |
| Hazard ratio | 1.00 | 0.90 | 0.83 | 0.83 | 0.85 | |
| 95% CI | (referent) | 0.78-1.03 | 0.73-0.95 | 0.72-0.94 | 0.77-0.94 | |
| P | (referent) | 0.12^{$} | 0.005^{#} | 0.004^{$} | 0.001 | |
| Myocardial | | | | | | |
| infarction, stroke, | | | | | | |
| or death from | | | | | | |
| any cause | | | | | | |
| Incidence rate, (N) | 5.56 | 5.17 | 4.77 | 4.88 | 4.93 | 0.015 |
| | (661) | (394) | (395) | (403) | (1192) | |
| Hazard ratio | 1.00 | 0.94 | 0.85 | 0.87 | 0.89 | |
| 95% CI | (referent) | 0.83-1.07 | 0.75-0.96 | 0.77-0.99 | 0.81-0.97 | |
| P | (referent) | 0.35 | 0.010 | 0.030 | 0.013 | |
| Myocardial | | | | | | |
| infarction | | | | | | |
| Incidence rate, (N) | 2.43 | 2.20 | 1.90 | 2.09 | 2.06 | 0.028 |
| | (292) | (169) | (159) | (174) | (502) | |
| Hazard ratio | 1.00 | 0.94 | 0.76 | 0.84 | 0.84 | |
| 95% CI | (referent) | 0.78-1.15 | 0.62-0.92 | 0.70-1.02 | 0.73-0.97 | |
| P | (referent) | 0.56 | 0.005 | 0.074 | 0.020 | |
| Hospitalization for | | | | | | |
| unstable angina | | | | | | |
| requiring urgent | | | | | | |
| revascularization | | | | | | |
| Incidence rate, (N) | 0.69 | 0.48 | 0.44 | 0.40 | 0.44 | 0.005 |
| | (85) | (38) | (38) | (34) | (110) | |
| Hazard ratio | 1.00 | 0.70 | 0.64 | 0.58 | 0.64 | |
| 95% CI | (referent) | 0.47-1.03 | 0.44-0.94 | 0.39-0.86 | 0.48-0.85 | |
| P | (referent) | 0.071 | 0.021 | 0.006 | 0.002 | |
| Any coronary | | | | | | |
| revascularization | | | | | | |
| Incidence rate, (N) | 3.61 | 2.53 | 2.49 | 2.56 | 2.53 | <0.001 |
| | (421) | (191) | (205) | (209) | (605) | |
| Hazard ratio | 1.00 | 0.72 | 0.68 | 0.70 | 0.70 | |
| 95% CI | (referent) | 0.60-0.86 | 0.58-0.81 | 0.59-0.83 | 0.62-0.79 | |
| P | (referent) | <0.001 | <0.001 | <0.001 | <0.001 | |
| Any Stroke | | | | | | |
| Incidence rate, (N) | 0.74 | 0.73 | 0.74 | 0.60 | 0.69 | 0.17 |
| | (92) | (58) | (63) | (51) | (172) | |
| Hazard ratio | 1.00 | 1.01 | 0.98 | 0.80 | 0.93 | |
| 95% CI | (referent) | 0.72-1.41 | 0.71-1.35 | 0.57-1.13 | 0.72-1.20 | |
| P | (referent) | 0.95 | 0.91 | 0.20 | 0.58 | |
| Cardiovascular | | | | | | |
| Death (confirmed) | | | | | | |
| Incidence rate, (N) | 1.44 | 1.18 | 1.26 | 1.33 | 1.26 | 0.76 |
| | (182) | (94) | (110) | (115) | (319) | |
| Hazard ratio | 1.00 | 0.80 | 0.88 | 0.93 | 0.87 | |
| 95% CI | (referent) | 0.62-1.03 | 0.70-1.12 | 0.74-1.18 | 0.73-1.05 | |
| P | (referent) | 0.083 | 0.30 | 0.55 | 0.15 | |
| Cardiovascular | | | | | | |
| death (or death | | | | | | |
| of unknown cause) | | | | | | |
| Incidence rate, (N) | 1.86 | 1.71 | 1.65 | 1.74 | 1.70 | 0.62 |
| | (235) | (137) | (144) | (151) | (432) | |
| Hazard ratio | 1.00 | 0.89 | 0.90 | 0.94 | 0.92 | |
| 95% CI | (referent) | 0.72-1.11 | 0.73-1.10 | 0.77-1.16 | 0.78-1.07 | |
| P | (referent) | 0.30 | 0.30 | 0.59 | 0.28 | |
| Non- | | | | | | |
| Cardiovascular | | | | | | |
| death (confirmed) | | | | | | |
| Incidence rate, (N) | 1.11 | 1.14 | 1.08 | 1.02 | 1.08 | 0.45 |
| | (140) | (91) | (94) | (88) | (273) | |
| Hazard ratio | 1.00 | 1.02 | 0.97 | 0.92 | 0.97 | |
| 95% CI | (referent) | 0.78-1.34 | 0.74-1.26 | 0.70-1.20 | 0.79-1.19 | |
| P | (referent) | 0.87 | 0.81 | 0.54 | 0.79 | |
| Total Mortality | | | | | | |
| Incidence rate, (N) | 2.97 | 2.85 | 2.73 | 2.76 | 2.78 | 0.39 |
| | (375) | (228) | (238) | (239) | (705) | |
| Hazard ratio | 1.00 | 0.94 | 0.92 | 0.94 | 0.94 | |
| 95% CI | (referent) | 0.80-1.11 | 0.78-1.09 | 0.80-1.10 | 0.83-1.06 | |
| P | (referent) | 0.48 | 0.33 | 0.42 | 0.31 | |

P values for trend, P values for the combination of all doses compared to placebo, and P values for all secondary end points other than the key secondary cardiovascular end point have not been adjusted for multiplicity.
*Primary end point = nonfatal myocardial infarction, nonfatal stroke, or cardiovascular death.
**Key secondary cardiovascular end point = nonfatal myocardial infarction, nonfatal stroke, hospitalization for unstable angina requiring unplanned revascularization, or cardiovascular death
^{#}Statistically significant compared to placebo, adjusted for multiplicity and accounting for two efficacy interim analyses, in accordance with the pre-specified closed-testing. The threshold P value for the primary end point for the 150 mg dose was 0.02115. The threshold P value for the key secondary cardiovascular end point for the 150 mg dose was 0.00529.
†Not statistically significant compared to placebo based on the prespecified closed-testing procedure. The threshold P value for the primary end point for the 50 mg dose was 0.02115. The threshold P value for the primary end point for the 300 mg dose was 0.01058.
^{$}Exploratory analyses.

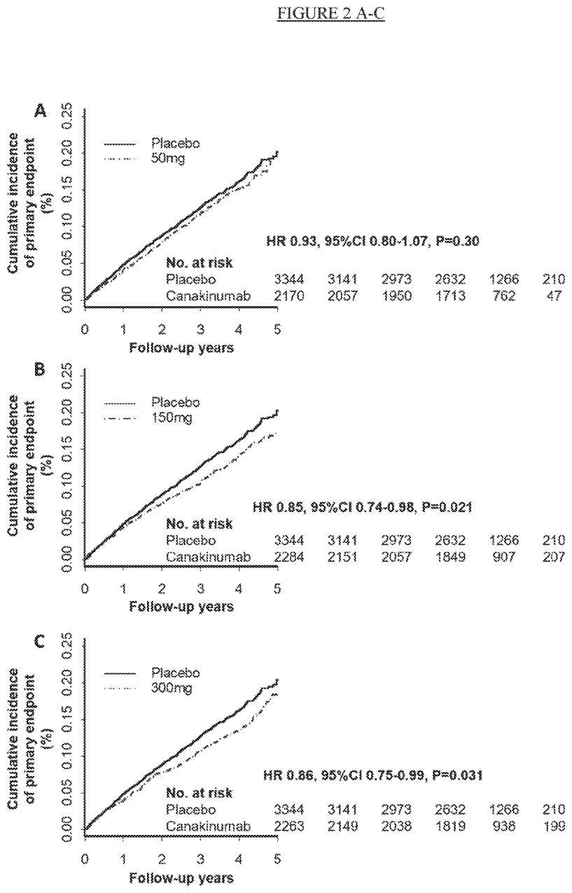

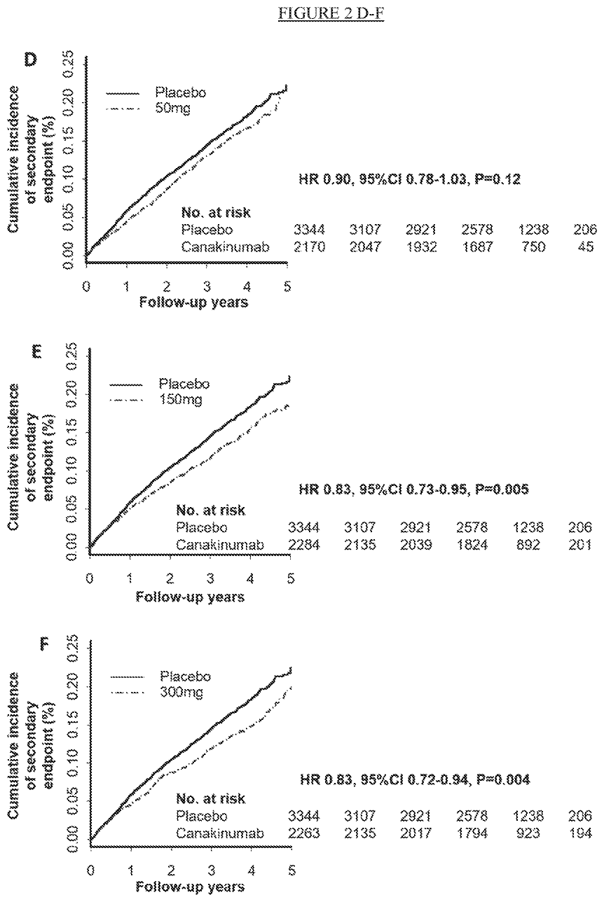

No significant effect was observed for the primary end point in the canakinumab 50 mg dose group compared to placebo (hazard ratio [HR] 0.93, P=0.30) (FIG. 2A). By contrast, a statistically significant effect for the primary end point was observed in the canakinumab 150 mg dose group (HR 0.85, P=0.02075, threshold P value 0.02115) (FIG. 2B). In the canakinumab 300 mg dose group, the hazard ratio was similar but the P value did not meet the prespecified significance threshold (HR 0.86, P=0.0314, threshold P value 0.01058) (FIG. 2C). The P value for trend across the active-dose groups compared to placebo was 0.020, and the P value for comparison of all doses combined versus placebo was 0.015 (both results not adjusted for multiple testing). Additionally, a subgroup of patients showing greater reductions in their hsCRP levels after treatment with canakinumab after 3 months show a statistically significant greater risk reduction in MACE compared to the overall treatment population. Patients responding with reductions in their hsCRP levels to <1.8 mg/L receiving 150 mg and 300 mg canakinumab, respectively, showed 24% and 22% relative risk reduction in MACE, respectively, based on causal inference analysis assuming exponential survival distribution, estimates based on 500 bootstrap samples (Table 8). Patients responding with reductions in their hsCRP levels to <1.5 mg/L receiving 150 mg and 300 mg canakinumab, respectively, showed a 26% and 27% relative risk reduction in MACE, respectively, based on causal inference analysis assuming exponential survival distribution, estimates based on 500 bootstrap samples (Table 8).

TABLE 8
hsCRP at three months responder analysis for risk of MACE

| | % of | Hazard ratio vs. | | |
|---|---|---|---|---|
| | overall | placebo (95% | NNT | NNT |
| Treatment strategy | population | CI) | 2 yrs | 3.8 yrs |
| All patients | | | | |
| Canakinumab 150/300 mg | 100% | 0.85 (0.76, 0.96) | 80 | 47 |
| combined | | | | |
| Canakinumab 300 mg | 100% | 0.86 (0.75, 0.99) | 83 | 49 |
| Canakinumab 150 mg | 100% | 0.85 (0.74, 0.98) | 78 | 46 |
| 3-month hsCRP non- | | | | |
| responders (≥1.8 mg/L)* | | | | |
| Canakinumab 150/300 mg | 45% | 0.91 (0.79, 1.03) | 98 | 63 |
| combined | | | | |
| Canakinumab 300 mg | 38% | 0.94 (0.78, 1.11) | 96 | 56 |
| Canakinumab 150 mg | 51% | 0.88 (0.75, 1.03) | 83 | 51 |
| 3-month hsCRP responders | | | | |
| (<1.8 mg/L)* | | | | |
| Canakinumab 150/300 mg | 55% | 0.77 (0.68, 0.89) | 52 | 31 |
| combined | | | | |
| Canakinumab 300 mg | 62% | 0.78 (0.66, 0.92) | 53 | 32 |
| Canakinumab 150 mg | 49% | 0.76 (0.64, 0.91) | 50 | 30 |
| 3-month hsCRP responders | | | | |
| (<1.5 mg/L)* | | | | |
| Canakinumab 150/300 mg | 49% | 0.73 (0.65, 0.85) | 45 | 26 |
| combined | | | | |
| Canakinumab 300 mg | 55% | 0.73 (0.61, 0.88) | 44 | 26 |
| Canakinumab 150 mg | 43% | 0.74 (0.59, 0.90) | 45 | 27 |

NNT: Number needed to treat to prevent one MACE event, excluding first dose
*Based on causal inference analysis assuming exponential survival distribution, estimates based on 500 bootstrap samples

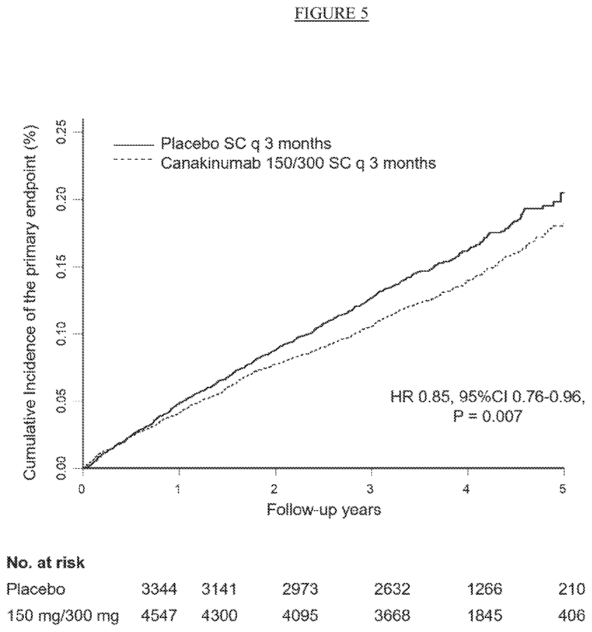

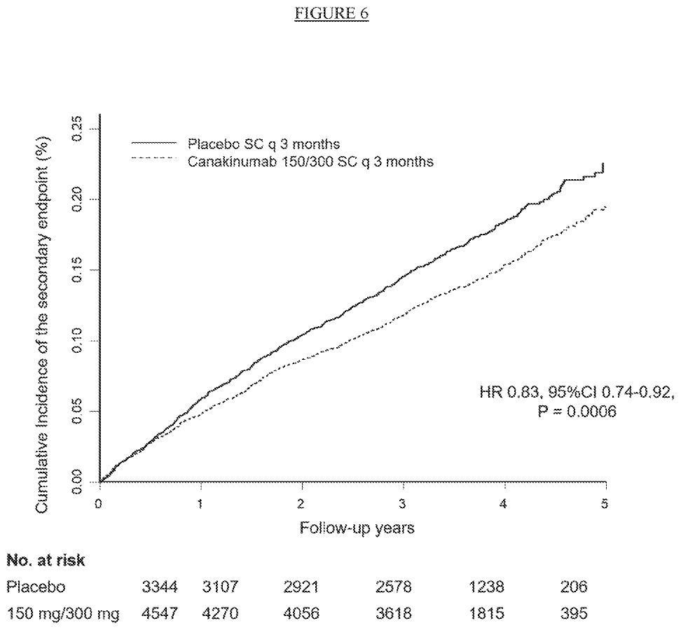

For the key secondary cardiovascular end point (which included the components of the primary end point plus hospitalization for unstable angina requiring urgent revascularization), incidence rates in the placebo, 50 mg, 150 mg, and 300 mg groups were 5.13, 4.56, 4.29, and 4.25 per 100 person-years, respectively (Table 7). For the canakinumab 150 mg dose (for which the P value met the significance threshold for the primary end point), the hazard ratio for the secondary cardiovascular endpoint was 0.83 (P=0.00525, threshold P value 0.00529) (FIG. 2D). According to the closed testing procedure, formal significance testing for the prespecified secondary end point was not performed for the 50 mg and 300 mg doses. The hazard ratios for these doses were 0.90 and 0.83, respectively (FIGS. 5 and 6). The P value for trend across the active-dose groups compared to placebo was 0.003, and the P value for comparison of all doses combined versus placebo was 0.001 (both results not adjusted for multiple testing).
Analyses of the additional secondary end points, and of the components of the primary and secondary end points, were not adjusted for multiple testing (Table 7). Nominally significant reductions were seen in myocardial infarction for the 150 mg dose of canakinumab; in hospitalization for unstable angina requiring urgent revascularization for the 150 mg and 300 mg doses; and in any coronary revascularization for all three doses. All-cause mortality was neutral in comparisons of all canakinumab doses to placebo (HR 0.94, 95% CI 0.83-1.06, P=0.31). In on-treatment analyses for the primary end point, the observed hazard ratios in the placebo, 50 mg, 150 mg, and 300 mg groups were 1.0, 0.90, 0.83, and 0.79 (P-trend across groups=0.003). In comparable analyses for the key secondary cardiovascular end point, the corresponding hazard ratios were 1.0, 0.88, 0.80, and 0.77 (P-trend across groups<0.001).

### Adverse Events and Other Clinical Outcomes.

Neutropenia was more common among those allocated to canakinumab and there was a statistically significant increase in fatal events attributed to infection or sepsis when the three canakinumab groups were pooled and compared to placebo (incidence rates 0.31 versus 0.18 per 100 person years, P=0.023) (Table 9). Participants succumbing to infection tended to be older and more likely to have diabetes. Six confirmed cases of tuberculosis occurred in the trial with similar rates in the canakinumab and placebo groups (0.06%); five cases occurred in India and one in Taiwan.

Thrombocytopenia was more common among those allocated to canakinumab, but no difference in hemorrhage was observed. No increase in injection site reactions was observed. Consistent with known effects of IL-1β inhibition, canakinumab resulted in significant reductions in reports of arthritis, gout, and osteoarthritis (Table 9). There was also a significant reduction in cancer mortality with canakinumab.

TABLE 9
Incidence rates (per 100-person years), number (N) of serious adverse events, and
selected on-treatment safety laboratory data (%, N), stratified by study group.

| | Canakinumab Dose (SC q 3 months) | | | | | | |
|---|---|---|---|---|---|---|---|
| | | | | | | P-value | P-value |
| | | | | | | for | for |
| | | | | | | trend | combined |
| | | | | | | across | dose |
| Adverse Event or | | | | | All | doses | groups |
| Laboratory | Placebo | 50 mg | 150 mg | 300 mg | doses | vs. | vs. |
| Parameter | n-3344 | N-2170 | N-2284 | N-2263 | N-6717 | placebo | placebo |
| Any SAE | 12.0 | 11.4 | 11.7 | 12.3 | 11.8 | 0.43 | 0.79 |
| | (1202) | (741) | (812) | (836) | (2389) | | |
| Any SAE infection | 2.86 | 3.03 | 3.13 | 3.25 | 3.14 | 0.12 | 0.14 |
| | (342) | (230) | (258) | (265) | (753) | | |
| Cellulitis | 0.24 | 0.24 | 0.37 | 0.41 | 0.34 | 0.0213 | 0.09 |
| | (30) | (19) | (32) | (35) | (86) | | |
| Pneumonia | 0.90 | 0.94 | 0.94 | 0.99 | 0.95 | 0.56 | 0.62 |
| | (112) | (74) | (80) | (84) | (238) | | |
| Urinary tract | 0.22 | 0.18 | 0.24 | 0.20 | 0.21 | 0.84 | 0.87 |
| | (27) | (14) | (21) | (17) | (52) | | |
| Opportunistic | 0.18 | 0.16 | 0.15 | 0.20 | 0.17 | 0.97 | 0.78 |
| infections+ | (23) | (13) | (13) | (17) | (43) | | |
| Pseudomembranous | 0.03 | 0.13 | 0.05 | 0.12 | 0.10 | 0.13 | 0.0302 |
| Colitis | (4) | (10) | (4) | (10) | (24) | | |
| Fatal | 0.18 | 0.31 | 0.28 | 0.34 | 0.31 | 0.09 | 0.0228 |
| infection/sepsis | (23) | (25) | (24) | (29) | (78) | | |
| Any malignancy++ | 1.88 | 1.85 | 1.69 | 1.72 | 1.75 | 0.31 | 0.38 |
| | (231) | (144) | (143) | (144) | (431) | | |
| Fatal | 0.64 | 0.55 | 0.50 | 0.31 | 0.45 | 0.0007 | 0.016 |
| malignancy++ | (81) | (44) | (44) | (27) | (115) | | |
| Other adverse | | | | | | | |
| events | | | | | | | |
| Injection site | 0.23 | 0.27 | 0.28 | 0.30 | 0.28 | 0.49 | 0.36 |
| reaction+ | (29) | (21) | (24) | (26) | (71) | | |
| Arthritis | 3.32 | 2.15 | 2.17 | 2.47 | 2.26 | 0.0020 | <0.0001 |
| | (385) | (164) | (180) | (201) | (545) | | |
| Osteoarthritis | 1.67 | 1.21 | 1.12 | 1.30 | 1.21 | 0.0393 | 0.0005 |
| | (202) | (94) | (95) | (109) | (298) | | |
| Gout | 0.80 | 0.43 | 0.35 | 0.37 | 0.38 | <0.0001 | <0.0001 |
| | (99) | (34) | (30) | (32) | (96) | | |
| Drug induced | 0.18 | 0.15 | 0.13 | 0.05 | 0.11 | 0.0039 | 0.0541 |
| liver injury (SAE)+ | (23) | (12) | (11) | (4) | (27) | | |
| Leukopenia | 0.24 | 0.30 | 0.37 | 0.52 | 0.40 | 0.002 | 0.013 |
| | (30) | (24) | (32) | (44) | (100) | | |
| Neutropenia | 0.06 | 0.05 | 0.07 | 0.18 | 0.10 | 0.014 | 0.17 |
| | (7) | (4) | (6) | (15) | (25) | | |
| Any Hemorrhage | 4.01 | 3.33 | 4.15 | 3.82 | 3.78 | 0.94 | 0.31 |
| | (462) | (249) | (327) | (301) | (877) | | |
| Thrombocytopenia | 0.43 | 0.56 | 0.54 | 0.71 | 0.60 | 0.022 | 0.031 |
| | (53) | (44) | (46) | (60) | (150) | | |
| Hepatic variable** | | | | | | | |
| ALT > 3x normal | 1.4 | 1.9 | 1.9 | 2.0 | 2.0 | 0.19 | 0.058 |
| %, (N) | (46) | (42) | (44) | (45) | (131) | | |
| AST > 3x normal | 1.1 | 1.5 | 1.5 | 1.5 | 1.5 | 0.30 | 0.11 |
| %, (N) | (36) | (32) | (35) | (34) | (101) | | |
| ALP > 3x normal | 0.4 | 0.5 | 0.4 | 0.5 | 0.5 | 0.67 | 0.82 |
| %, (N) | (15) | (11) | (10) | (12) | (33) | | |
| Bilirubin >2x | 0.8 | 1.0 | 0.7 | 0.7 | 0.8 | 0.34 | 0.83 |
| normal %, (N) | (26) | (21) | (15) | (15) | (51) | | |

Data are shown as incidence rates per 100 person-years (with number of patients with event) for adverse events and as percentages of patients with the condition (with number of patients) for hepatic variables to facilitate the comparison of rates between groups. All adverse event categories are based on standardized Medical Dictionary for Regulatory Activities (MedDRA), version 20.0, queries or classification levels, except those otherwise indicated.
SAE = serious adverse event;
ALT = alanine aminotransferase;
AST = aspartate transaminase;
ALP = alkaline phosphatase
+Sponsor categorization of adverse events of special interest
++Included are malignancies adjudicated by the Cancer Endpoint Adjudication Committee
**Hepatic variable-percent of patients with condition (No.)

CANTOS was designed to test directly the inflammatory hypothesis of atherothrombosis. In this trial, among patients with a prior history of myocardial infarction, hsCRP levels and IL-6 levels were significantly reduced by canakinumab, with no reduction in lipid levels. While the 50 mg dose of canakinumab did not have a statistically significant effect on the primary cardiovascular end point compared to placebo, participants in the 150 mg dose group experienced relative hazard reductions of 15% for the primary end point (from 4.50 to 3.86 events per 100 person-years) and 17% for the key secondary cardiovascular end point (from 5.13 to 4.29 events per 100 person-years). The P values for both of these end points met pre-specified multiplicity-adjusted thresholds for statistical significance. Although the hazard reductions for the 300 mg dose group were similar to those for the 150 mg dose group, the prespecified thresholds for statistical significance were not met for this group. Both a pooled analysis of all canakinumab doses and a trend analysis, however, suggested a beneficial effect of canakinumab on cardiovascular outcomes. Specific targeting of IL-1β as a cytokine-based therapy for the secondary prevention of atherosclerotic events rests on several observations. The pro-inflammatory cytokine IL-1β plays multiple roles in atherothrombotic plaque development including induction of procoagulant activity, promotion of monocyte and leucocyte adhesion to vascular endothelial cells, and the growth of vascular smooth muscle cells (Dinarello C A et al, Nat Rev Drug Discov. 2012; 11(8):633-52; Dinarello C A. Blood. 2011; 117(14):3720-32; Libby P et al, Am J Pathol. 1986; 124(2):179-85). In mice, deficiency of IL-1β reduces lesion formation, while in cholesterol-fed pigs, exposure to exogenous IL-1β increases intimal medial thickening (Kirii H et al, Arterioscler Thromb Vasc Biol. 2003; 23(4):656-60; Shimokawa H et al, J Clin Invest. 1996; 97(3):769-76). The Nod-like receptor protein 3 (NLRP3) inflammasome activates IL-1β, a process promoted by cholesterol crystals, neutrophil extracellular traps, local hypoxia, and atheroprone flow (Duewell P et al, Nature. 2010; 464(7293):1357-61; Rajamaki K et al, PLoS One. 2010; 5(7):e11765; Xiao H et al, Circulation. 2013; 128(6):632-42; Folco E J et al, Circ Res. 2014; 115(10):875-83. This activation of IL-1ß stimulates the downstream IL-6 receptor signaling pathway, implicated by Mendelian randomization studies as a potential causal pathway for atherothrombosis (Hingorani A D et al, Lancet. 2012; 379(9822):1214-24; Sarwar N et al, Lancet. 2012; 379(9822):1205-13. Most recently, parabiotic mouse studies (Sager H B et al, Circulation. 2015; 132(20):1880-90) and studies of clonal hematopoiesis (Fuster J J et al, Science. 2017; 355(6327):842-7; Jaiswal S et al, N Engl J Med. 2017; 377(2):111-21) have implicated IL-1β in processes by which bone marrow activation accelerates atherosclerosis. Further, expression of specific inflammasome gene modules impacting IL-1β associates with all-cause mortality and increased atherosclerosis in the elderly (Furman D et al, Nat Med. 2017; 23(2):174-84).

Although the patients in CANTOS had generally well-controlled levels of LDL cholesterol, placebo event rates were high, with a cumulative incidence of over 20% at five years. Our data thus affirm that statin-treated patients with residual inflammatory risk as assessed by baseline hsCRP greater than 2 mg/L have future event rates at least as high as, if not higher than, statin-treated patients with residual risk due to LDL cholesterol. These two patient groups may differ and may require personalized approaches to treatment (Ridker P M. Eur Heart J. 2016; 37(22):1720-2). Despite the fact that no reduction in cholesterol levels occurred, the magnitude of effect on cardiovascular events with canakinumab (given every 3 months) was comparable to that associated with monoclonal antibodies targeting PCSK9 (given every 2 to 4 weeks) (Sabatine M S et al, N Engl J Med. 2017; 376(18): 1713-22; Ridker P M et al, N Engl J Med. 2017; 376(16): 1527-39). Yet inhibition of IL-1β is a narrowly focused intervention that represents only one of many potential anti-inflammatory pathways that might serve as targets for atheroprotection (Morton A C et al, Eur Heart J. 2015; 36(6):377-84; Van Tassell B W et al, Circulation. 2013; 128(17): 1910-23; Ridker P M et al, Eur Heart J. 2014; 35(27): 1782-91). We observed a statistically significant increase in fatal infection and sepsis with canakinumab, as well as a reduction in platelet counts with no increase in bleeding. By contrast, there was a significant reduction in cancer mortality among those allocated to canakinumab, a finding consistent with experimental data relating IL-1 to the progression and invasiveness of certain tumors, in particular lung cancer (Ridker P M et al, Lancet. 2017; 390(10105):1833-42; Apte R N et al, Cancer Metastasis Rev. 2006; 25(3):387-408; Grivennikov S I et al, Lancet 2000; 355:735-740). There was no significant difference between treatment groups in all-cause mortality. No significant hepatic toxicity was noted. The beneficial effects of canakinumab observed for arthritis, gout, and osteoarthritis are consistent with well-described effects of the IL-1 and IL-6 pathways in these disorders. In conclusion, in CANTOS, patients with a prior history of myocardial infarction and hsCRP levels of 2 mg/L or greater were randomized to one of three doses of canakinumab or placebo. Canakinumab significantly reduced hsCRP levels without reducing LDL cholesterol, HDL cholesterol and triglycerides and the 150 mg dose significantly reduced the incidence of recurrent cardiovascular events whilst having an acceptable levels of side effects.

### High-Responder Analysis

Blood samples were obtained from all trial participants in the canakinumab and placebo groups at randomization and among 9,534 participants (94.8%) at 3 months, just prior to repeat canakinumab (or placebo) injection. All baseline and 3 month samples underwent assay for hsCRP and lipid levels in a central laboratory.

For the high-responder analysis, it was first ascertained whether the overall effect of canakinumab on future cardiovascular event rates was modified by common baseline clinical characteristics including age, gender, diabetes, smoking status, body mass index, hsCRP or lipid levels. Trial participants allocated to canakinumab were divided into two groups according to whether the level of hsCRP at 3 months was less than, or equal to or greater than 2 mg/L, a commonly used clinical cutpoint for hsCRP. Chi-square tests were used to assess for significant differences between these two groups for categorical variables and Wilcoxon rank sum tests for continuous variables.

On a per-protocol pre-specified basis, Cox proportional-hazards models stratified by time since index myocardial infarction and trial part were used to estimate relative hazards for major adverse cardiovascular events in these two groups, compared with those allocated placebo. Similar analyses compared the outcomes of cardiovascular mortality and all-cause mortality, and major adverse effects such as infection. To address issues of confounding, multivariable modeling was used to adjust for baseline characteristics known to modestly impact on hsCRP including age, gender, smoking status, hypertension, diabetes, and body mass index. The multivariable models additionally adjusted for baseline hsCRP and LDL-C. As an internal check to ensure the validity of this approach, the above process was repeated alternatively dividing the cohort according to whether the level of hsCRP at 3 months was less than, or equal to or greater than the study on-treatment median value of 1.8 mg/L; on the basis of tertiles of on-treatment hsCRP at 3 months (rather than medians); on the basis of achieving reductions in hsCRP at 3 months of greater than or less than 50%; and on the basis of achieving greater or less than the percent median reduction in hsCRP. Similar analyses were performed at individual canakinumab dose levels to eliminate the potential for confounding on this basis.

In all instances, Cox proportional hazards models were used to estimate hazard ratios (HR) comparing the different on-treatment canakinumab groups to placebo. P-values for the test of trend were calculated across these three groups scored as 0, 1, or 2. Kaplan-Meier curves were constructed to visually evaluate any differences between groups. As an alternative method to evaluate potential differences in on-treatment groups, a causal inference analysis was conducted which compared potential outcomes of individual canakinumab treated participants had they counterfactually been treated with placebo. This latter analysis was again performed at individual canakinumab dose levels to eliminate the potential for confounding on this basis. To provide an overall assessment of clinical efficacy, the number needed to treat (NNT) over five years for the endpoint inclusive of myocardial infarction, stroke, coronary revascularization, or death from any cause was computed as the reciprocal of the absolute difference between risks in canakinumab versus placebo treated patients based on Kaplan-Meier estimates of risk. Estimates were calculated for the cohort as a whole and separately among those who did or did not achieve hsCRP levels below 2 mg/L.

All P-values are two-sided, and all confidence intervals computed at the 95% level. The trial is registered at ClinicalTrial.gov, NCT01327846.

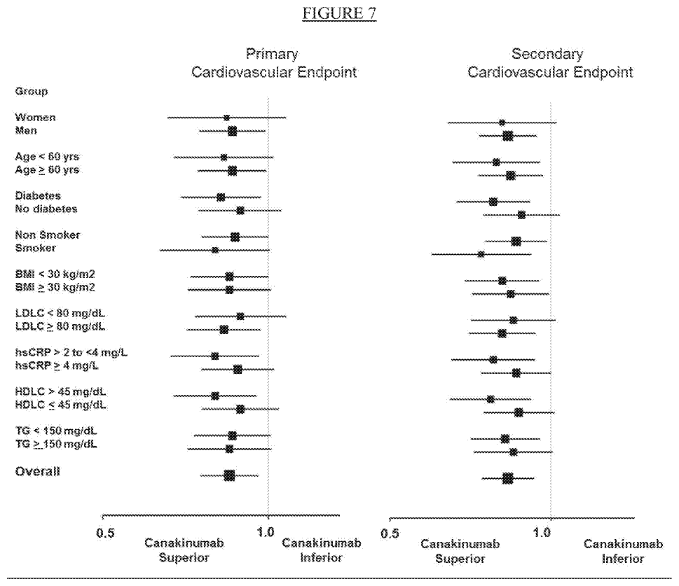

It was initially addressed whether any baseline clinical characteristic of the CANTOS population might have modified the effect of canakinumab on clinical outcomes. However, all major clinical subgroups benefited from canakinumab for both the primary and secondary cardiovascular endpoints (FIG. 7). Canakinumab had similar efficacy among those with LDLC levels above and below 80 mg/dL (2.06 mmol/L), the approximate trial median level at study entry (FIG. 7).

To begin the evaluation of whether on-treatment hsCRP levels can predict clinical outcomes with canakinumab, it was examined how those who achieved lower levels differed from those who did not. Table 10 shows baseline characteristics of the study population in the placebo group and in the combined canakinumab groups according to whether the on-treatment hsCRP level was below versus at or above 2 mg/L when measured at 3 months (prior to receiving the next dose). As anticipated, hsCRP levels were lower at baseline among participants who subsequently achieved 3 month levels below 2 mg/L compared to those who did not. The proportions of individuals achieving on treatment hsCRP levels below 2 mg/L was 22%, 44%, 55%, and 65% in the placebo and canakinumab 50 mg, 150 mg, and 300 mg groups, respectively (P<0.0001).

TABLE 10
Baseline clinical characteristics of the CANTOS population in the placebo group
and in the canakinumab groups according to achieved concentrations of hsCRP above or
below the median 3-month on-treatment value of 2.0 mg/L. Data shown are medians
(interquartile range) for continuous variables, and N (percentages) for categorical variables.

| | | Canakinumab, | Canakinumab, |
|---|---|---|---|
| | | 3-month on | 3-month on |
| | | treatment | treatment |
| | Placebo | hsCRP ≥ 2.0 mg/L | hsCRP < 2.0 mg/L |
| | (N = 3182) | (N = 2868) | (N = 3484) |
| Age (yr) | 61.0(54.0, 68.0) | 61.0(55.0, 68.0) | 61.0(54.0, 68.0) |
| Female sex | 817(25.7) | 753(26.3) | 864(24.8) |
| Current smoking | 722(22.7) | 795(27.7) | 717(20.6) |
| Body mass index (kg/m²) | 29.7(26.6, 33.9) | 30.5(27.0, 34.7) | 29.6(26.4, 33.1) |
| Waist circumference (cm) | 104.0(96.0, 114.3) | 106.0(97.0, 116.8) | 103.0(95.0, 112.0) |
| Hypertension | 2514(79.0) | 2328(81.2) | 2747(78.8) |
| Diabetes | 1265(39.8) | 1229(42.9) | 1307(37.5) |
| Qualifying myocardial | | | |
| infarction | | | |
| STEMI | 1714(53.9) | 1531(53.4) | 1960(56.3) |
| Non-STEMI | 1076(33.8) | 983(34.3) | 1143(32.8) |
| Unknown/missing | 392(12.3) | 354(12.3) | 381(10.9) |
| History of PCI | 2099(66.0) | 1920(67.0) | 2375(68.2) |
| History of CABG | 453(14.2) | 436(15.2) | 458(13.2) |
| History of congestive | 684(21.5) | 662(23.1) | 699(20.1) |
| heart failure | | | |
| Lipid lowering therapy | 2980(93.7) | 2647(92.5) | 3284(94.3) |
| Renin-angiotensin | 2527(79.6) | 2263(79.2) | 2787(80.2) |
| inhibitors | | | |
| Anti-ischemia agents* | 2929(92.0) | 2624(91.7) | 3172(91.1) |
| hsCRP (mg/L) | 4.10(2.75, 6.85) | 5.55(3.60, 9.25) | 3.40(2.45, 5.20) |
| Interleukin-6 (ng/L) | 2.59(1.79, 4.03) | 3.02(2.02, 4.86) | 2.27(1.59, 3.43) |
| Total cholesterol (mg/dL) | 160.5(137.0, 189.0) | 162.0(137.3, 191.0) | 157.8(135.0, 186.0) |
| LDL cholesterol (mg/dL) | 82.8(64.6, 107.1) | 83.5(65.0, 109.0) | 81.2(62.3, 104.9) |
| HDL cholesterol (mg/dL) | 44.5(37.1, 52.6) | 43.0(36.0, 52.0) | 44.1(37.1, 53.0) |
| Triglycerides (mg/dL) | 139.1(100.1, 195.0) | 141.7(103.6, 201.9) | 136.4(101.0, 190.0) |
| eGFR (mL/min/1.73 m²) | 79.0(65.0, 93.0) | 79.0(64.0, 93.0) | 78.0(65.0, 92.0) |
| Randomized to | | 1155(40.3) | 905(26.0) |
| canakinumab 50 mg | | | |
| Randomized to | | 977(34.1) | 1186(34.0) |
| canakinumab 150 mg | | | |
| Randomized to | | 736(25.7) | 1393(40.0) |
| canakinumab 300 mg | | | |

*Anti-ischemia agents are defined as beta-blocking agents, nitrates, or calcium channel-blocking agents.
Yr = years, STEMI = ST-segment elevation myocardial infarction, PCI = Percutaneous coronary intervention, CABG = Coronary artery bypass grafting, eGFR = estimated glomerular filtration rate

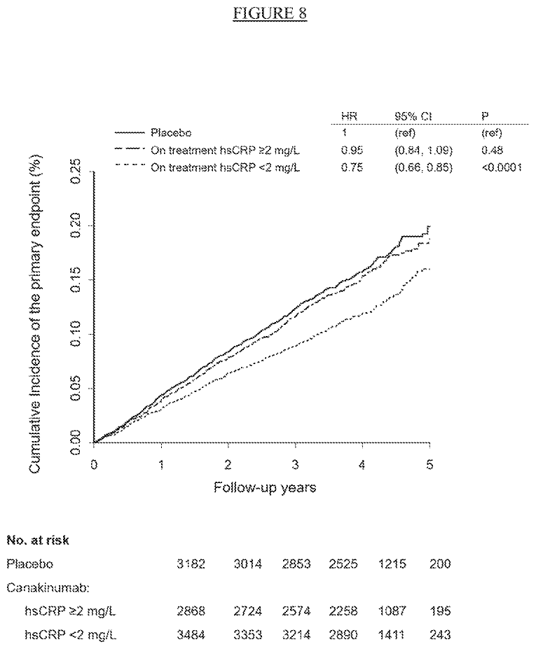

In univariate analyses, the magnitude of decrease in hsCRP with canakinumab related directly to the magnitude of clinical benefit associated with canakinumab treatment (Table 11). Compared to placebo, participants allocated to any dose of canakinumab who had 3-month hsCRP levels equal to or above 2 mg/L did not have a statistically significant reduction in clinical events; for this group (N=2868), the hazard ratio compared to placebo for the trial primary endpoint was 0.95 (95% CI 0.84-1.09, P=0.48). By contrast, trial participants allocated to canakinumab who did achieve a 3-month hsCRP less than 2 mg/L had a highly significant and much larger 25 percent reduction in risk (hazard ratio for the primary endpoint=0.75, 95% CI 0.65-0.85, P<0.0001) (FIG. 8). In this analysis, the P-value for the test of trend across hsCRP strata was <0.0001. These data correspond to incidence rates for the primary endpoint of 4.39, 4.20, and 3.28 events per 100 person years in the placebo group and in the combined canakinumab groups that did not and did achieve hsCRP levels below 2 mg/L, respectively.

A series of sensitivity analyses were performed to evaluate the robustness of these findings and to address whether any potential confounding factors had magnitudes of effect on achieved hsCRP similar to that of canakinumab itself. First, baseline hsCRP and LDL cholesterol level were adjusted for, as well as clinical characteristics known to modestly impact on hsCRP (including age, gender, smoking status, hypertension, diabetes, and body mass index). In these multivariable analyses, the calculated hazard ratios for major adverse cardiovascular events among those treated with canakinumab who had hsCRP levels at 3 months below or above 2 mg/L (adjusted HRs 0.75 and 0.90 respectively) were minimally changed from those observed in the univariate analysis (unadjusted HRs 0.75 and 0.95, respectively) (Table 11).

TABLE 11
Incidence rates (per 100 person years) and hazard ratios for the primary pre-
specified cardiovascular endpoint of major adverse cardiovascular events (nonfatal
myocardial infarction, nonfatal stroke, or cardiovascular death) in CANTOS, according to
the magnitude or levels of on-treatment reduction in hsCRP achieved at 3 months among
those allocated to canakinumab.

| Treatment group, on- | | | | |
|---|---|---|---|---|
| treatment hsCRP | | Incidence | | HR^{adjusted}** (95% CI), |
| threshold | N | Rate*(n) | HR (95% CI), P | P |
| Placebo | 3182 | 4.39 (500) | 1.0 | 1.0 |
| Canakinumab, hsCRP ≥ | 2868 | 4.20 (431) | 0.95 (0.84-1.09), | 0.90 (0.79-1.02), 0.11 |
| 2 mg/L | | | 0.48 | |
| Canakinumab, hsCRP < | 3484 | 3.28 (421) | 0.75 (0.66-0.85), | 0.75 (0.66-0.85), |
| 2 mg/L | | | <0.0001 | <0.0001 |
| P-value for trend across | | | <0.0001 | <0.0001 |
| categories | | | | |
| Placebo | 3182 | 4.39 (500) | 1.0 | 1.0 |
| Canakinumab, hsCRP ≥ | 3193 | 4.19 (480) | 0.95 (0.84-1.08), | 0.90 (0.79-1.02), |
| median value (1.8 | | | 0.47 | 0.10 |
| mg/L) | | | | |
| Canakinumab, hsCRP < | 3159 | 3.19 (372) | 0.73 (0.63-0.83), | 0.73 (0.64-0.84), |
| median value (1.8 | | | <0.0001 | <0.0001 |
| mg/L) | | | | |
| P-value for trend across | | | <0.0001 | <0.0001 |
| categories | | | | |
| Placebo | 3182 | 4.39 (500) | 1.0 | 1.0 |
| Canakinumab, hsCRP | 2090 | 4.36 (325) | 0.99 (0.86-1.14, | 0.93 (0.80-1.07), 0.29 |
| top tertile*** | | | 0.93 | |
| Canakinumab, hsCRP | 2044 | 3.66 (273) | 0.83 (0.72-0.96), | 0.80 (0.69-0.93), |
| middle tertile | | | 0.0136 | 0.0037 |
| Canakinumab, hsCRP | 2218 | 3.09 (254) | 0.71 (0.61-0.82), | 0.73 (0.62-0.85), |
| lowest tertile | | | <0.0001 | <0.0001 |
| P-value for trend across | | | <0.0001 | <0.0001 |
| categories | | | | |
| Placebo | 3182 | 4.39 (500) | 1.0 | 1.0 |
| Canakinumab, hsCRP | 2537 | 3.82 (347) | 0.87 (0.76-1.00), | 0.87 (0.76-1.00), |
| reduction ≤ 50% | | | 0.0461 | 0.0459 |
| Canakinumab, hsCRP | 3746 | 3.58 (494) | 0.81 (0.72-0.92), | 0.81 (0.71-0.91), |
| reduction > 50% | | | 0.0012 | 0.0007 |
| P-value for trend across | | | 0.0013 | 0.0008 |
| categories | | | | |
| Placebo | 3182 | 4.39 (500) | 1.0 | 1.0 |
| Canakinumab, hsCRP ≤ | 3142 | 3.77 (426) | 0.86 (0.75-0.98), | 0.86 (0.75-0.98), |
| median % | | | 0.0203 | 0.0228 |
| reduction**** | | | | |
| Canakinumab, hsCRP > | 3141 | 3.59 (415) | 0.82 (0.72-0.93), | 0.80 (0.70-0.92), |
| median % reduction | | | 0.0021 | 0.0010 |
| P-value for trend across | | | 0.0019 | 0.0010 |
| categories | | | | |

*per 100 person-years of exposure.
**Covariates included in the adjusted multivariable model include age, gender, smoking status, hypertension, diabetes, body mass index, baseline level of hsCRP, and baseline level of LDL cholesterol.
***Tertile cutpoints for on-treatment hsCRP levels at 3 months were >2.6 mg/L, >1.2 to 2.6 mg/L, and < or equal to 1.2 mg/L.
****The median % reduction in hsCRP at 3-months was −58.14.

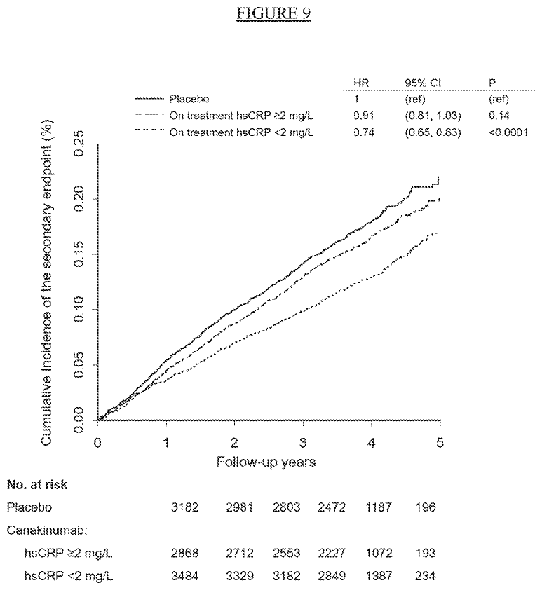

Second, the analysis was repeated using the trial pre-specified secondary cardiovascular endpoint which included nonfatal myocardial infarction, nonfatal stroke, hospitalization for unstable angina requiring urgent revascularization, or cardiovascular death. Compared to placebo, participants allocated to any dose of canakinumab who did not achieve a 3-month hsCRP less than 2 mg/L did not have a statistically significant reduction in the secondary trial endpoint; for this group, the adjusted hazard ratio compared to placebo for the secondary trial endpoint was 0.91 (95% CI 0.81-1.03, P=0.14). By contrast, trial participants allocated to canakinumab who did achieve a 3-month hsCRP of less than 2 mg/L had a statistically significant 26 percent reduction in risk (adjusted hazard ratio for the primary endpoint of 0.74, 95% CI 0.65-0.83, P<0.0001) (FIG. 9). In this analysis, the P-value for the test of trend across hsCRP strata was <0.0001 and the P-value for the comparison between active treatment groups was 0.0011. These data correspond to incidence rates for the secondary trial endpoint of 5.02, 4.57, and 3.70 events per 100 person years in the placebo group and in the combined canakinumab groups that did not and did achieve hsCRP levels below 2 mg/L, respectively.

Third, the analysis was repeated using the median 3-month on-treatment level of hsCRP observed among those allocated to any dose of canakinumab (1.8 mg/L rather than the clinical cut-point of 2 mg/L) and observed similar effects in both univariable and multivariable analyses (Table 11).

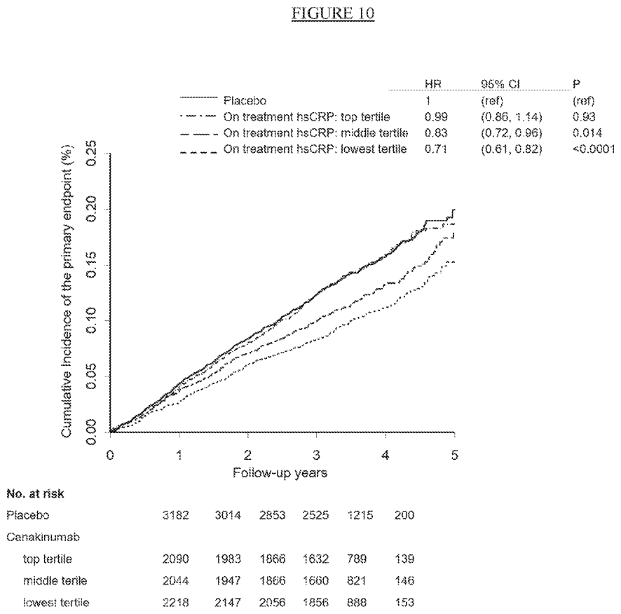

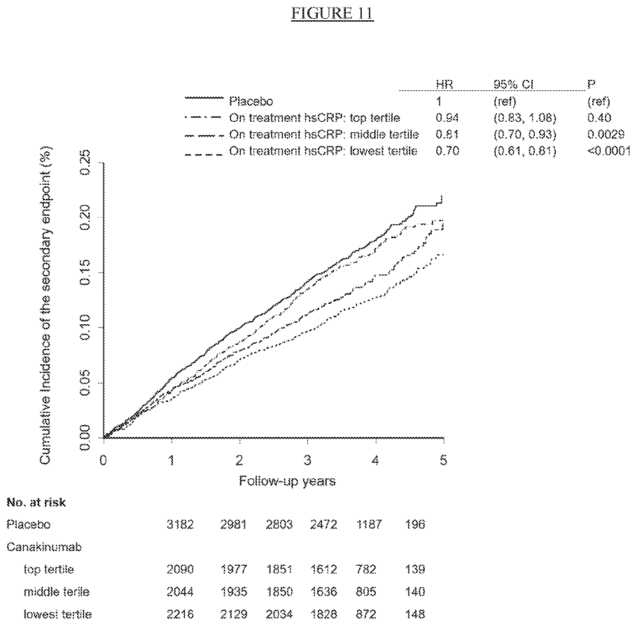

Fourth, the analysis of the primary and secondary cardiovascular endpoints was repeated across tertiles of on treatment hsCRP levels (rather than median levels) at 3 months. Effects were again similar in both univariable and multivariable analyses with the greatest reductions in risk for both the primary and secondary cardiovascular endpoints accruing among those with the greatest magnitude of hsCRP reduction (Table 11, FIGS. 10 and 11).

Fifth, repeated analyses that used an on-treatment target of less than or equal to or greater than a 50% reduction in hsCRP at 3-months yielded similar results (P-trend across groups 0.0008) albeit with mild attenuation between the two active groups (Table 11).

Sixth, the analyses were repeated using an on-treatment target less than, or equal to or greater than the median percent reduction in hsCRP, and again observed similar results (P-trend across groups 0.0010) (Table 11).

Seventh, the analyses were repeated for three additional cardiovascular endpoints pre-specified in the CANTOS protocol. Cardiovascular death (fully adjusted 0.69, 95% CI 0.56-0.85, P=0.0004) and all-cause mortality (fully adjusted HR 0.69, 95% CI 0.58-0.81, P<0.0001) both fell significantly among those who achieved on-treatment hsCRP levels less than 2 mg/L. A similar benefit was observed for the additional protocol pre-specified endpoint of myocardial infarction, stroke, or death from any cause among those who achieved on-treatment hsCRP less than 2 mg/L (HR 0.73, 95% CI 0.65-0.82, P<0.0001). By contrast, no significant effects were observed for any of these additional endpoints among those treated with canakinumab who did not achieve hsCRP levels below this threshold (Table 12).

TABLE 12
Incidence rates and adjusted hazard ratios for additional
pre-specified cardiovascular endpoints in CANTOS, according
to on-treatment hsCRP levels at 3 months less than 2 mg/L or
greater than or equal to 2 mg/L.

| | | Canakinumab | Canakinumab | |
|---|---|---|---|---|
| | | 3-month | 3-month | |
| | Placebo | hsCRP ≥ 2 | hsCRP < | P-value |
| | (N = | mg/L | 2 mg/L | across |
| Clinical Outcome | 3182) | (N = 2868) | (N = 3484) | doses |
| Myocardial | | | | |
| infarction, | | | | |
| stroke, or death | | | | |
| from any cause | | | | |
| Incidence rate, (N) | 5.39 (614) | 5.38 (553) | 3.96 (508) | |
| Hazard | 1.00 | 0.93 | 0.73 | |
| ratio^{fully adjusted} | | | | |
| 95% CI | (referent) | 0.83-1.05 | 0.65-0.82 | |
| P | (referent) | 0.25 | <0.0001 | <0.0001 |
| Cardiovascular death | | | | |
| Incidence rate, (N) | 1.74 (211) | 1.83 (198) | 1.22 (164) | |
| Hazard | 1.00 | 0.99 | 0.69 | |
| ratio^{fully adjusted} | | | | |
| 95% CI | (referent) | 0.82-1.21 | 0.56-0.85 | |
| P | (referent) | 0.95 | 0.0004 | 0.0004 |
| All-Cause Mortality | | | | |
| Incidence rate, (N) | 2.79 (338) | 3.14 (339) | 1.96 (264) | |
| Hazard | 1.00 | 1.05 | 0.69 | |
| ratio^{fully adjusted} | | | | |
| 95% CI | (referent) | 0.90-1.22 | 0.58-0.81 | |
| P | (referent) | 0.56 | <0.0001 | <0.0001 |

Incidence rates are calculated per 100 person-years of exposure. Covariates included in the multivariable fully adjusted data include age, gender, smoking status, hypertension, diabetes, body mass index, baseline level of hsCRP, and baseline level of LDL cholesterol.

Eighth, to assess the potential for residual confounding by randomized drug allocation, separate analyses were performed for each individual canakinumab dose. Compared to placebo, the multivariable adjusted hazard ratios for the primary endpoint among those who achieved 3 month on-treatment hsCRP levels below 2 mg/L were 0.78 (95% CI 0.63-0.96, P=0.0195), 0.75 (95% CI 0.62-0.91, P=0.0028), and 0.74 (95% CI 0.62-0.88, P=0.0009) in the canakinumab 50 mg, 150 mg, and 300 mg groups, respectively. By contrast, no significant benefits of canakinumab were observed at any individual dose among those who did not achieve 3 month hsCRP levels below 2 mg/L. In multivariable adjusted models further adjusted simultaneously for all three dose groups, the adjusted hazard ratio for the primary endpoint among those who achieved 3 month on-treatment hsCRP levels below 2 mg/L was 0.79 (95% CI 0.66-0.94, P=0.0065), while those who did not achieve hsCRP levels at 3 months below this threshold had no significant benefit (HR=0.94, 95% CI 0.80-1.10, P=0.41).

A causal inference analysis was additionally conducted in which potential outcomes were modeled using baseline covariates (age, gender, body mass index, smoking status, diabetes, blood pressure, hsCRP, total and HDL cholesterol, glomerular filtration rate, prior history and timing of vascular disease) for individual canakinumab treated patients had they counterfactually been allocated to placebo, and then compared the modeled effects to observed effects. In this alternative analysis approach designed to address the estimation of treatment effect of canakinumab in patients who had achieved target levels of hsCRP, highly similar results at individual doses of canakinumab were observed. For example, for those treated with 150 mg canakinumab who achieved a 3-month hsCRP below the trial median value of 2 mg/L, the relative hazard ratio for major adverse cardiovascular events compared with their outcome had they counterfactually been assigned to placebo was 0.76 (95% CI 0.64-0.91) whereas for those who achieved a 3-month hsCRP≥2 mg/L, the comparable relative hazard was 0.90 (95% CI 0.75-1.07). Similarly, for those treated with 300 mg canakinumab who achieved a 3-month hsCRP below 2 mg/L, the relative hazard ratio for major adverse cardiovascular events compared with their outcome had they counterfactually been assigned to placebo was 0.80 (95% CI 0.69-0.96) whereas for those who achieved a 3-month hsCRP≥2 mg/L, the comparable relative hazard was 0.93 (95% CI 0.74-1.04).

The calculated number needed to treat (NNT) over five years for myocardial infarction, stroke, coronary revascularization, or death from any cause for the CANTOS cohort as a whole was 24. Among those with on-treatment hsCRP values below 2 mg/L, the 5-year NNT estimate was 16. The 5-year NNT estimate was 57 for those who did not achieve on-treatment hsCRP levels below this threshold.

Virtually identical results were obtained for the above analyses using on-treatment levels of interleukin-6 (IL-6) rather than on-treatment levels of hsCRP.

Canakinumab did not associate with any adverse hepatic, renal, or hemorrhagic effects, and this safety profile was also observed in analyses stratified by on-treatment levels of hsCRP. Overall in CANTOS, canakinumab was associated with an increase in fatal infection, but this latter effect was not dose-dependent. In on-treatment analyses, the incidence rate of fatal infection among canakinumab treated patients who achieved a 3-month hsCRP less than 2 mg/L was 0.27 per 100 person-years and the incidence rate of fatal infection among canakinumab treated patients who did not achieve this level of hsCRP was 0.35 per 100-person years. While these rates were both higher than the placebo rate of 0.18 per 100-person years, there was no statistically significant difference in incidence rates for fatal infection comparing the two canakinumab groups defined by on-treatment levels of hsCRP at 3 months (P=0.33). Power was limited to detect differences, however, as the total number of fatal infections was small.

The analysis of 9,534 post-myocardial infarction patients in CANTOS demonstrates that the magnitude of reduction in hsCRP following the first dose of canakinumab relates directly to the magnitude of long-term clinical benefit for incident cardiovascular events, cardiovascular death, and all-cause mortality. The differential outcomes observed in CANTOS on the basis of achieved hsCRP concentration were robust to the choice of on-treatment measures (values above or below thresholds defined by medians, tertiles, percentage reductions, or commonly used clinical cutpoints), were minimally affected by adjustment for baseline clinical characteristics known to impact on achieved hsCRP levels, were observed at all individual dose levels, and were additionally observed in a causal inference analysis. Conversely, this analysis indicated no substantial differences between achieved hsCRP and safety outcomes, including for rates of infection.

This is of interest for several reasons. First, the data for canakinumab and on-treatment hsCRP is analogous to prior work from lipid-lowering trials and on-treatment levels of LDL-C. Further, as most CANTOS participants were already on high intensity statin therapy, the current data provide independent support for the general concept that achieving the “dual goals” of inflammation reduction as well as cholesterol reduction provides the greatest clinical benefits with regard to atherothrombotic prevention.

Second, the data have practical implications for the potential use of canakinumab as an adjunctive therapy for post-myocardial infarction patients. As shown in FIG. 7, all common clinical subgroups achieved similar relative risk reductions with canakinumab with no evidence of statistically significant heterogeneity. Thus, other than high absolute risk, there does not appear to be a simple way to maximize canakinumab efficacy using baseline clinical characteristics of the CANTOS cohort.

By contrast, as shown here, the magnitude of benefit associated with canakinumab for cardiovascular events (as well as all-cause mortality) related directly to the magnitude of hsCRP reduction achieved after a single dose. Indeed, those who did not achieve substantial reductions in hsCRP had only small and non-significant benefits (FIGS. 8-11). These data support the use of an on-treatment measure of hsCRP as a simple clinical mechanism to differentiate between candidates for sustained canakinumab treatment and individuals much less likely to benefit from continued treatment.

Application of this clinical strategy would ensure a favorable benefit to risk ratio for canakinumab and simultaneously improve cost-effectiveness. For example, the overall 5-year number needed to treat in CANTOS for the endpoint inclusive of myocardial infarction, stroke, any coronary revascularization, or death from any cause is 24. However, if only those with on-treatment hsCRP values below 2 mg/L are analyzed, the 5-year number needed to treat is 16. This contrasts to a 5-year number needed to treat of 57 for those who do not achieve hsCRP levels below 2 mg/L.

Differences in the data appeared to be modest as multivariate adjustment for a large range of baseline clinical characteristics including hsCRP and canakinumab dose had minimal impact on the findings. Also, almost identical differential effects were observed in a causal inference analysis in which those treated with canakinumab were modeled as having counterfactually been treated with placebo. While no analysis can fully eliminate residual confounding, the fact that different forms of adjustment led to similar results and had relatively small impact on the overall finding is consistent with the hypothesis that the magnitude of direct biologic effect of canakinumab on hsCRP is far larger than the magnitude of effect associated with other potential clinical variables.

As shown in the pre-specified on-treatment analyses, a greater proportion of individuals allocated to 300 mg of canakinumab than to 150 mg of canakinumab achieved hsCRP reductions at 3 months below thresholds associated with the greatest clinical benefit. Thus, for individual patients where variability in drug response may exist, the availability of both a 150 mg and a 300 mg dose may broaden the clinical utility of canakinumab.

In sum, in these pre-specified analyses from the multinational CANTOS trial, the magnitude of reduction in hsCRP, in the absence of any change in LDL-C, was strongly related to the magnitude of cardiovascular event reduction and all-cause mortality reduction following canakinumab therapy.

### Causal Interference Analysis

A separate causal inference analysis was conducted: the method estimates average treatment effect in the subgroup of patients who achieve hsCRP levels below the specified target at 3-months following treatment with canakinumab. The estimation of these potential outcomes differs from the multivariable adjustment described above in that it allows to ascertain the average treatment comparison in the population of patients who would achieve on treatment hsCRP values below the target levels of interest. In the application of the causal inference analysis the number of patients included in the analyses was expanded to encompass all 10,009 patients who were alive at the time of the 3-month assessment and could have provided a sample by relying on multiple imputation of the missing hsCRP values in order to avoid introducing bias by excluding patients who might have contributed events to the analysis but were initially excluded due to the unavailability of an assayed sample.

For canakinumab treated patients the treatment effect as the hazard rate of occurrence of the endpoint of interest (MACE) was observed, but for placebo treated patients their hsCRP levels under treatment with canakinumab are unknown. Hence the placebo survival of canakinumab “responder” patients is derived, i.e., canakinumab responder patients counterfactually treated with placebo, by deriving the average survival of placebo patients predicted from the covariate values of canakinumab responder patients. The baseline covariates are those that are useful for predicting hsCRP response below a certain target level when treated with canakinumab: baseline hsCRP, Body-Mass Index (BMI), the SMART risk score established by the European Society for Cardiology (Dorresteijn, J. A. N. et al, Heart. 2013; 99(12):866-72), LDL-C and baseline statin dose. The hazard rates for these two groups are derived: canakinumab treated patient using observed risk, and the average over the covariate weighted survival of the placebo patients who would have been responders when treated with canakinumab. Hazard rates were then obtained using non-parametric or semiparametric models (Cox regression) stratified by time since qualifying MI for survival and then estimating the hazards. The causal inference approach does not provide p-values, only bounds calculated as the quantiles corresponding to the usual two-sided 95% intervals from a bootstrap resampling procedure applied. These hazard rates were used to derive hazard ratios, with confidence bounds being derived from 3,000 bootstrap iterations, which included accounting for the uncertainty of multiply imputed hsCRP values for patients not having a laboratory value at 3 months and who did not have a death date prior to or on Day 92.

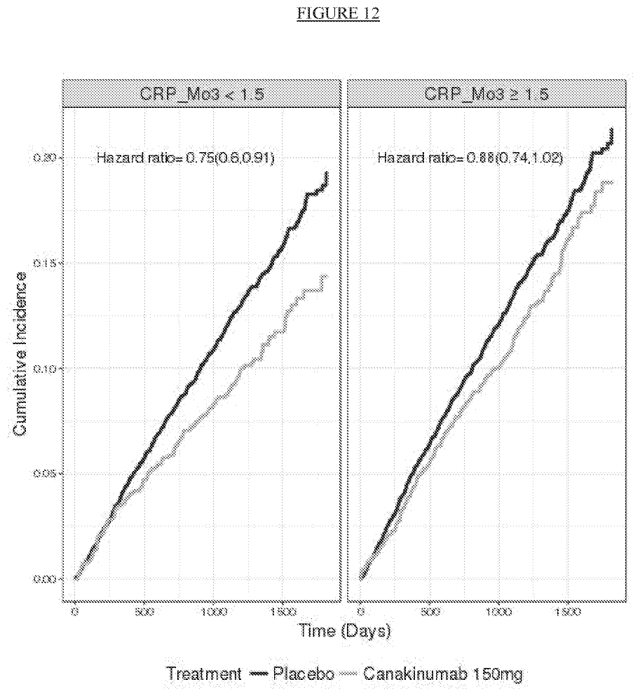

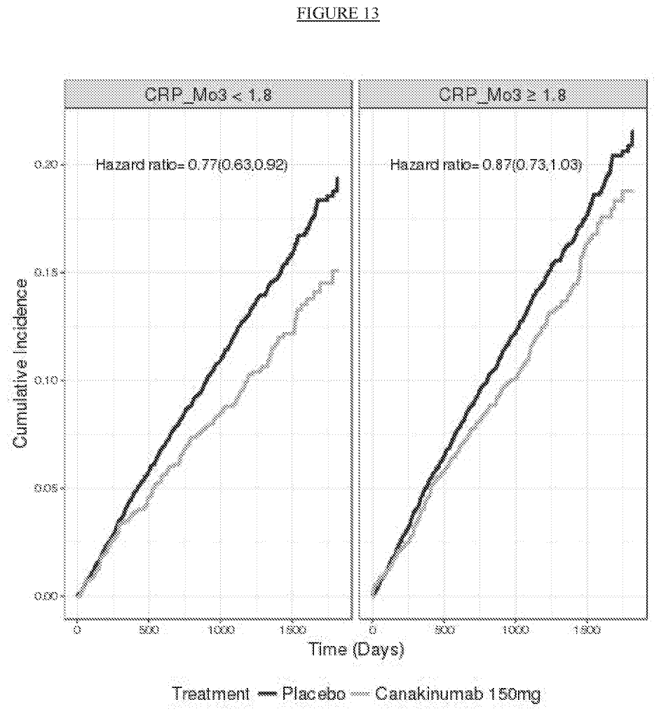

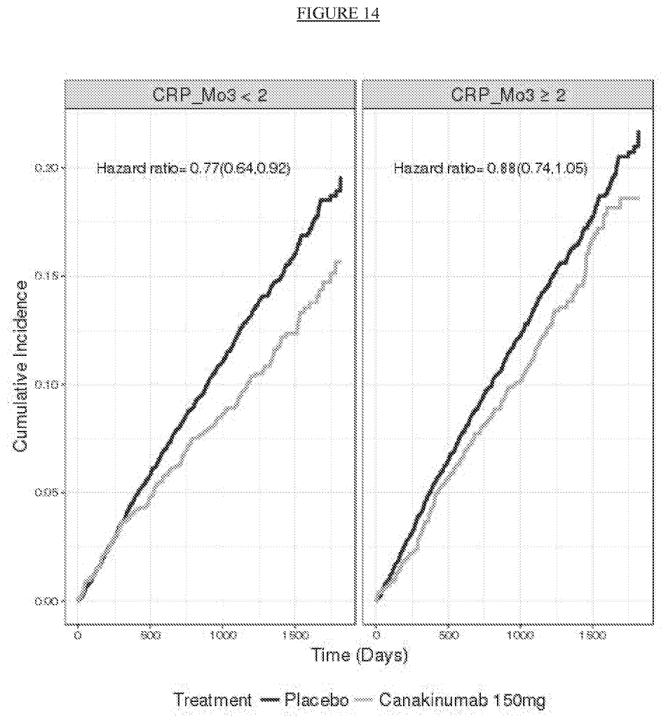

For those treated with 150 mg canakinumab who achieved a 3-month hsCRP below 2 mg/L, the relative HR for major adverse cardiovascular events (MACE) compared with their outcome had they counterfactually been assigned to placebo was 0.77 (95% CI 0.64-0.92) whereas for those who achieved a 3-month hsCRP≥2 mg/L, the comparable relative hazard was 0.88 (95% CI 0.74-1.05) (Table 13 and FIG. 14). Similarly, for those who achieved a 3-month hsCRP below the trial median value of 1.8 mg/L, the relative hazard ratio for major adverse cardiovascular events compared with their outcome had they counterfactually been assigned to placebo was 0.77 (95% CI 0.63-0.92) whereas for those who achieved a 3-month hsCRP≥1.8 mg/L, the comparable relative hazard was 0.87 (95% CI 0.73-1.03) (Table 13 and FIG. 13). For those treated with 150 mg canakinumab who achieved a 3-month hsCRP below 1.5 mg/L, the relative HR for major adverse cardiovascular events (MACE) compared with their outcome had they counterfactually been assigned to placebo was 0.75 (95% CI 0.6-0.91) whereas for those who achieved a 3-month hsCRP≥1.5 mg/L, the comparable relative hazard was 0.88 (95% CI 0.74-1.02) (Table 13 and FIG. 12). As shown here, the magnitude of benefit associated with canakinumab for major adverse cardiovascular events relate directly to the achieved hsCRP levels after a single dose of canakinumab.

TABLE 13
Causal inference analysis used for model-based quantification of
treatment effect for the primary endpoint MACE (nonfatal
myocardial infarction, nonfatal stroke, or cardiovascular death)
in CANTOS, according to indicated on-treatment hsCRP levels
at 3 months following treatment 150 mg canakinumab

| | Target hsCRP | | Hazard Ratio | | |
|---|---|---|---|---|---|
| Endpoint | value at | Treatment | Estimate | Lower | Upper |
| MACE | <1.5 | 150 mg | 0.75 | 0.6 | 0.91 |
| MACE | ≥1.5 | 150 mg | 0.88 | 0.74 | 1.02 |
| MACE | <1.8 | 150 mg | 0.77 | 0.63 | 0.92 |
| MACE | ≥1.8 | 150 mg | 0.87 | 0.73 | 1.03 |
| MACE | <2 | 150 mg | 0.77 | 0.64 | 0.92 |
| MACE | ≥2 | 150 mg | 0.88 | 0.74 | 1.05 |

1. A method for reducing the risk of or preventing recurrent cardiovascular (CV) events in a patient that has suffered myocardial infarction (MI), comprising administering about 150 mg to about 300 mg of canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab.

2. A method for reducing the risk of or preventing recurrent cardiovascular (CV) events in a patient that has suffered myocardial infarction (MI), comprising administering about 150 mg to about 300 mg of canakinumab, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient will continue to receive about 150 mg to about 300 mg canakinumab approximately every 3 months, provided said patient has a reduced hsCRP level of <2 mg/L assessed approximately 3 months after first administration of canakinumab.

3. The method of claim 1 or 2, comprising administering 150 mg or 300 mg canakinumab.

4. The method according to any of the preceding claims, comprising administering 150 mg canakinumab.

5. A method for reducing the risk of or preventing recurrent cardiovascular (CV) events in a patient that has suffered myocardial infarction (MI), comprising administering about 150 mg canakinumab approximately every 3 months, wherein said patient has a high sensitivity C-reactive protein (hsCRP) level of ≥2 mg/L assessed at least 28 days after MI and before first administration of canakinumab, and wherein canakinumab is administered at the earliest 30 days after MI, and wherein said patient has a reduced hsCRP level of <2 mg/L assessed approximately 3 months after first administration of canakinumab.

6. The method according to any of the preceding claims, wherein the reduced level of hsCRP assessed approximately 3 months after first administration of canakinumab is <1.8 mg/L.

7. The method according to any of the preceding claims, wherein the reduced level of hsCRP assessed approximately 3 months after first administration of canakinumab is <1.5 mg/L.

8. The method according to any of the preceding claims, wherein said recurrent CV event is selected from non-fatal MI, non-fatal stroke, cardiovascular (CV) death and hospitalization for unstable angina requiring unplanned revascularization.

9. The method according to any one of claims 1 to 8, wherein said recurrent CV event is selected from non-fatal MI, non-fatal stroke and cardiovascular (CV) death.

10. The method according to any one of claims 1 to 8, wherein said recurrent CV event is non-fatal MI or cardiovascular (CV) death.

11. The method according to any one of claims 1 to 8, wherein said recurrent CV event is non-fatal MI.

12. The method according to any one of claims 1 to 8, wherein said recurrent CV event is hospitalization for unstable angina requiring unplanned revascularization.

13. The method according to any of the preceding claims, wherein said patient is concomitantly receiving standard of care treatment for reducing the risk of or preventing recurrent CV events.

14. Canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

15. Canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, provided said patient has a reduced hsCRP level of <2 mg/L assessed approximately 3 months after first administration of canakinumab.

16. Use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg to about 300 mg of canakinumab is administered to the patient at the earliest 30 days after MI, and
iii) wherein said patient will continue to receive about 150 mg to about 300 mg canakinumab approximately every 3 months, and
iv) wherein said patient has a reduced hsCRP level of <2 mg/L assessed approximately 3 months after first administration of canakinumab.

17. Use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and wherein
ii) about 150 mg to about 300 mg of canakinumab is administered to the patient at the earliest 30 days after MI, and wherein
iii) said patient will continue to receive about 150 mg to about 300 mg canakinumab approximately every 3 months, provided said patient has a reduced hsCRP level of <2 mg/L assessed approximately 3 months after first administration of canakinumab.

18. The use according to any one of claims 14 to 17, comprising administering 150 mg or 300 mg canakinumab.

19. The use according to any one of claims 14 to 18, comprising administering 150 mg canakinumab.

20. Canakinumab for use in reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab approximately every 3 months, and
iv) wherein said patient has a reduced hsCRP level of <2 mg/L assessed approximately 3 months after first administration of canakinumab.

21. Use of canakinumab for the manufacture of a medicament for reducing the risk of or preventing recurrent cardiovascular (CV) events in a patient that has suffered myocardial infarction (MI),
i) wherein said patient has a high sensitivity C-reactive protein (hsCRP) level of ≥2 mg/L assessed at least 28 days after MI and before first administration of canakinumab, and
ii) wherein about 150 mg canakinumab is administered to the patient at the earliest 30 days after MI, and
iii) wherein said patient will continue to receive about 150 mg canakinumab approximately every 3 months, and wherein said patient has a reduced hsCRP level of <2 mg/L assessed approximately 3 months after first administration of canakinumab.

22. The use according to any one of claims 14 to 21, wherein the reduced level of hsCRP is <1.8 mg/L assessed approximately 3 months after first administration of canakinumab.

23. The use according to any one of claims 14 to 22, wherein the reduced level of hsCRP is <1.5 mg/L assessed approximately 3 months after first administration of canakinumab.

24. Canakinumab for use according to any one of claims 14 to 23, wherein said recurrent CV event is selected from non-fatal MI, non-fatal stroke, cardiovascular (CV) death or hospitalization for unstable angina requiring unplanned revascularization.

25. Canakinumab for use according to any one of claims 14 to 23, wherein said recurrent CV event is selected from non-fatal MI or non-fatal stroke or cardiovascular (CV) death.

26. Canakinumab for use according to any one of claims 14 to 23, wherein said recurrent CV event is non-fatal MI or cardiovascular (CV) death.

27. Canakinumab for use according to any one of claims 14 to 23, wherein said recurrent CV event is non-fatal MI.

28. Canakinumab for use according to any one of claims 14 to 23, wherein said recurrent CV event is hospitalization for unstable angina requiring unplanned revascularization.

29. Canakinumab for use according to any one of claims 14 to 28, wherein said patient is concomitantly receiving standard of care treatment for reducing the risk of or preventing recurrent CV events.
